(19) United States
(12) Patent Application Publication
(10) Pub. No.: US 2020/0109362 A1
(43) Pub. Date: Apr. 9, 2020

(54) METHODS FOR CELL PROLIFERATION AND TOXICITY TESTING

(71) Applicant: Massachusetts Institute of Technology, Cambridge, MA (US)

(72) Inventors: Bevin P. Engelward, Lexington, MA (US); Le P. Ngo, Boston, MA (US); Tze Khee Chan, Singapore (SG); Jing Ge, Somerville, MA (US)

(73) Assignee: Massachusetts Institute of Technology, Cambridge, MA (US)

(21) Appl. No.: 16/312,249

(22) PCT Filed: Jun. 21, 2017
(86) PCT No.: PCT/US2017/038624

§ 371 Date: Dec. 20, 2018

Related U.S. Application Data
(60) Provisional application No. 62/357,617, filed on Jul. 1, 2016.
(60) Provisional application No. 62/352,605, filed on Jun. 21, 2016.

(51) Int. Cl. C12M 1/34; C12M 1/32; G01N 33/569; B01L 3/00; G01N 33/50; C12N 5/00

(52) U.S. Cl. CPC C12M 41/46; C12M 23/12; G01N 33/56966; B01L 3/502715; B01L 2300/0829; B01L 3/5085; C12N 5/0018; B01L 2300/069; G01N 33/5023

(57) ABSTRACT

Provided herein are methods and devices for measuring and monitoring proliferation and toxicity in vitro.

### RELATED APPLICATIONS

This application is a national stage filing under 35 U.S.C. § 371 of International Application No. PCT/US2017/038624, entitled “METHODS FOR CELL PROLIFERATION AND TOXICITY TESTING,” filed Jun. 21, 2017, which claims the benefit under 35 U.S.C. § 119(e) of U.S. Provisional Application Ser. No. 62/352,605 entitled “TOXCHIP” filed on Jun. 21, 2016, and U.S. Provisional Application Ser. No. 62/357,617 entitled “USE OF MICROCOLONY SIZE DISTRIBUTION FOR HIGH THROUGHPUT TOXICITY TESTING” filed on Jul. 1, 2016, the entire contents of each of which are incorporated by reference herein.

### FEDERALLY SPONSORED RESEARCH

This invention was made with U.S. Government support under Grant No. R44-ES024698 awarded by the National Institutes of Health. The Government has certain rights in this invention.

### BACKGROUND OF INVENTION

Various assays are known and have been routinely used to monitor cell survival and/or the effects of drugs on cells. These assays include clonogenic assays and metabolic activity assays. Some measure the ability of a cell to give rise to a daughter cell, such as the clonogenic assay, while others measure metabolic activity. The clonogenic assay is the most sensitive cell viability assay and provides a direct measure of the ability of cells to divide, but it is time and labor intensive, typically yielding results only after weeks, and it can be costly. It also requires relatively large dishes and volumes of media. Metabolic activity assays such as XTT, MTT, and CellTiter-Glo (CTG) are commonly used high-throughput assays, but they are not as sensitive and/or robust as the clonogenic assay. Because metabolic activity is an indirect measure of cell viability, these assays are also less reliable since their readouts can be susceptible to culture conditions that affect cellular activity without causing cell death. Therefore, these assays typically require the use of another assay, such as the clonogenic assay, to validate their results.

### SUMMARY OF INVENTION

Provided herein are novel and improved methods, assays, devices and systems for performing cell proliferation and/or cell toxicity assays.

Provided in one aspect, is a method for monitoring cell growth in vitro comprising loading cells in a plurality of microwells, culturing the cells under conditions and for a time sufficient for cell growth and/or proliferation, thereby forming a microcolony in each microwell, staining the microcolonies with a membrane-permeable DNA-specific fluorescent dye, and imaging the microcolonies, thereby obtaining total fluorescent intensity per microcolony. The conditions and time sufficient for cell growth and/or proliferation will be governed by the cell type being used and the extent of cell growth required. Typically, the method measures cell proliferation, since mere cell maintenance without division may appear as background growth. Examples of membrane-permeable DNA-specific fluorescent dyes include but are not limited to Vybrant DyeCycle dyes, acridine orange, SYTO nucleic acid stains from ThermoFisher, Hoechst

In some embodiments, the microwells are defined by a semi-solid matrix. In some embodiments, the microwells are defined by a solid matrix. In some embodiments, the semi-solid matrix is agarose. In some embodiments, the agarose is normal melting point agarose. In some embodiments, the semi-solid matrix is a biologically compatible polymer. In some embodiments, the microwells are defined by a matrix comprising a hydrogel or polydimethylsiloxane.

In some embodiments, the plurality of microwells is provided in a fixed array of microwells. In some embodiments, the plurality of microwells is physically partitioned from other pluralities of microwells. In some embodiments, the plurality of microwells is physically partitioned by a macrowell of a bottomless 96 well plate. The plurality of microwells may be physically partitioned by virtually anything that can physically penetrate the matrix and create isolated regions (or partitions). Other “n-well” bottomless plates would work as well, including 24-well, 12-well, and 4-well plates.

In some embodiments, the number of cells initially loaded into the microwells is not uniform across the plurality. In some embodiments, the number of cells initially loaded into microwells is not uniform between pluralities.

In some embodiments, cells in the microcolonies are not lysed before being stained.

In some embodiments, the cells are loaded into the microwells by gravity. In some embodiments, the number of cells initially loaded into each microwell is in the range of 0-7 cells. In other embodiments, the number of cells initially loaded into each microwell is in the range of 0-10, 1-20, 0-50, 0-100, 0-500, 0-1000, 0-2000, or more including 5-10, 10-20, 20-5-, 50-100, 200-500, 500-1000, and 1000-2000. Thus, cell numbers in the single digits, double digits (tens), or hundreds, or thousands may be initially loaded into microwells provided the microwells can accommodate such numbers.

In some embodiments, the time sufficient for cell growth and/or proliferation is 1 day, 2 days, 3 days, 4 days, 5 days, 6 days, 7 days, or longer including weeks and months.

In some embodiments, the microcolonies are imaged using a fluorescent microscope such as an epifluorescent microscope. In some embodiments, the microcolonies are imaged using a detector that measures luminescence or radioactivity, if the DNA is stained with luminescent or radioactive labels instead of a fluorescent dye.

In some embodiments, a plurality of microcolonies are simultaneously imaged. In some embodiments, a plurality of microcolonies are consecutively imaged.

In some embodiments, 50-100, or 100-200, or more microcolonies are imaged, simultaneously or consecutively. The number of microcolonies that can be imaged simultaneously depends in part on the sensor size of the microscope's camera and also on the distance between microwells in the fixed array.

In some embodiments, the plurality of microwells are exposed to an agent after the cells are plated. In some embodiments, the agent is a candidate growth-modifying agent or cytotoxic agent. As used herein, a candidate agent is an agent that is being tested for one or more particular activities, such as in this case growth-modifying (e.g., stimulation or inhibition of cell proliferation) activity or cytotoxic activity.

In some embodiments, the plurality of microwells is provided in a chip that comprises other pluralities of microwells, each plurality physically partitioned from other pluralities.

In sonic embodiments, a second plurality of microwells is not exposed to the agent.

In sonic embodiments, cells loaded into the microwells are layered with low melting point agarose or any biocompatible polymer with tunable (adjustable) rigidity, such as for example hydrogels.

In some embodiments, cells loaded into the microwells are layered with one or more extracellular matrix components, which is/are then layered with low melting point agarose or other biocompatible polymer. The extracellular components may be collagen, fibronectin, gelatin, mixtures thereof, and the like.

In some embodiments, the cells are adherent cells. In some embodiments, the cells are non-adherent cells (otherwise referred to as suspension cells).

In some embodiments, the cells are a cell line. In some embodiments, the cells are cancer cells. In some embodiments, the cells are a cancer cell line. In some embodiments, the cells are normal cells.

In some embodiments, the cells are human cells. In some embodiments, the cells are prokaryotic cells such as bacterial cells.

In some embodiments, the total fluorescent intensity per microcolony comprises fluorescence intensity from live and dead cells in the microcolony.

In some embodiments, the microcolonies are non-clonal cell clusters each comprising 1-2000 cells.

In some embodiments, the DNA-specific fluorescent dye is a Vybrant DyeCycle dye, acridine orange, a SYTO nucleic acid stain, or a Hoechst stain.

The foregoing embodiments should be understood to apply equally to the various aspects described herein unless explicitly stated otherwise. For brevity, they will not be repeated for each aspect of this disclosure.

Provided in another aspect is a method for monitoring cytotoxic or growth inhibition effect of a compound on a population of cells comprising loading cells in a plurality of semi-solid microwells, exposing the cells to a candidate cytotoxic or growth inhibiting compound for a limited time, culturing the cells under conditions and for a time sufficient for cell growth and/or proliferation, thereby forming microcolonies in each microwell, staining the microcolonies with a membrane-permeable DNA-specific fluorescent dye, imaging the microcolonies, thereby obtaining total fluorescent intensity per microcolony, and measuring proliferation in the plurality of semi-solid microwells after exposure to the candidate cytotoxic or growth modifying (inhibiting or stimulating) compound.

In sonic embodiments, measuring proliferation comprises measuring proliferation fraction, in some embodiments, measuring proliferation comprises measuring total proliferation fraction fluorescence. In some embodiments, measuring proliferation comprises analysis of microcolony size distribution.

In some embodiments, the microwells in a plurality comprise a non-uniform number of cells. In some embodiments, the microwells in a plurality each comprise 0-7 cells, 0-10 cells, 0-50 cells, 0-100 cells, 0-500 cells, 0-1000 cells or more. In some embodiments, the microcolonies are non-clonal cell clusters each comprising 1-2000 cells.

In some embodiments, the cytotoxic or growth inhibiting effect of a number of different compounds is monitored simultaneously using different pluralities of microwells provided in a single fixed array.

In some embodiments, the microcolonies are stained without prior lysis of the cells.

Provided in another aspect is a method for measuring proliferation in a cell population comprising providing a fixed array of microwells arranged as physically partitioned pluralities of microwells, loading cells into the microwells by gravity, wherein the number of cells between microwells of a plurality is not uniform, exposing at least one plurality to a candidate cytotoxic or growth modifying compound, wherein at least one other plurality is not exposed to the candidate cytotoxic or growth modifying compound, culturing the cells under conditions and for a time sufficient for cell growth and/or proliferation to form a microcolony per microwell, measuring total DNA per microwell without lysing cells within the microwells, and measuring proliferation fraction of treated cells relative to untreated cells.

In some embodiments, the method further comprises measuring total proliferation fraction fluorescence of treated cells and untreated cells.

In some embodiments, the microwells are semi-solid microwells.

In some embodiments, the total number of cells in each plurality is approximately equal between pluralities.

In some embodiments, the total number of cells in each plurality is different between pluralities.

Provided in another aspect is a fixed array of semi-solid microwells with pluralities of microwells physically partitioned from each other, wherein the microwells within a plurality comprise a non-uniform number of cells, and wherein one or more cells are overlaid with an extracellular matrix and a semi-solid matrix, optionally wherein total cells between pluralities is approximately uniform.

Provided in another aspect is a fixed array of microwells with pluralities of microwells physically partitioned from each other, and a cell membrane-permeable DNA-specific fluorescent dye, wherein the microwells within a plurality comprise a non-uniform number of non-lysed cells, wherein total cells between pluralities is approximately uniform.

In some embodiments, one or more cells are fixed in microwells by an overlay of a matrix such as a semi-solid matrix. In some embodiments, the overlay of a semi-solid matrix is an overlay of low melting point agarose.

In some embodiments, each plurality comprises about 50, about 100, about 200 or about 500 microwells.

In some embodiments, the cells are adherent cells. In some embodiments, the cells are non-adherent cells.

In some embodiments, the microwells are semi-solid microwells and may comprise a semi-solid matrix and optionally a culture medium such as a complete culture medium. In some embodiments, the semi-solid matrix is normal melting point agarose. In some embodiments, the microwells are solid matrix microwells.

In some embodiments, the fixed array is immersed in culture medium.

In some embodiments, the microwells within a plurality comprise 0-7, or 0-10 or 0-50 or 0-100 or 0-500 or 0-1000 cells per microwell.

In some embodiments, the device further comprises a cell membrane permeable DNA-specific fluorescent dye. In some embodiments, the cells have not been lysed.

These and other aspects and embodiments of the invention will be described in greater detail herein.

### BRIEF DESCRIPTION OF DRAWINGS

FIG. 1. Microfabricated mold creates precision microwells that can be loaded with single or groups of cells.

FIG. 2. Example of a macrowell former. A bottomless 96-well plate can he pressed on top of the microwell array to create 96 physically isolated macrowells. Each macrowell contains a plurality of microwells

FIGS. 3A-3B. FIG. 3A. Exemplary steps in micropatterning cells in a microcolony chip (μCC). 1. PDMS stamp with microposts is pressed into molten agarose. 2. Agarose is allowed to cool and solidify. 3. Stamp is lifted off to reveal patterned microwells on agarose chip. 4. Cell suspension is placed directly onto agarose chip. 5. Cells settle into microwells via gravity. Excess cells are washed off to reveal micropatterned cells. 6. Cells are kept in microwells by an overlay layer of 0.3% low-melting point agarose. FIG. 3B. Phase-contrast pictures of patterned TK6 microcolonies taken at 40× magnification. Left: empty agarose microwell array. Day 0: micropatterned cells after loading. Day 1-Day 4: growth of patterned microcolonies during 4 days in culture.

FIG. 4. Colony formation on ToxChip at 24 hours and 96 hours after seeding with TK6 non-adherent (or suspension cells, as the terms are used interchangeably herein) and HeLa adherent cells.

FIG. 5. Colony detection and colony size quantification. A measurement that correlates with cells per colony is obtained by analyzing the total (green) fluorescence intensity of a microcolony.

FIG. 6. Total integrated fluorescence intensity per microcolony (FM) distribution of TK6 cells cultured on ToxChip for over 120 hours. Cells were stained with SYBR.

FIG. 7. Strong linear relationship between F/M and number of cells per microwell.

FIG. 8A. Integrated Fluorescence per Microcolony (F/M) without using a background correction step. (Left) Bright-field image of a TK6 microcolony (more than 50 cells) on ToxChip after 4 days in culture. (Middle) DNA fluorescent image of a different TK6 microcolony on the same ToxChip (DNA stained with Vybrant® DyeCycle Green). (Right) Fluorescence intensity plot of the TK6 microcolony in the middle image. The F/M is calculated as the total area.

FIG. 8B. Integrated fluorescence per microcolony (F/M) using background correction step. Left: Phase-contrast image of a TK6 microcolony (more than 60 cells) on μCC after 4 days in culture. Middle: DNA fluorescent image of a different TK6 microcolony (DNA stained with Vybrant® DyeCycle Green). Right: Fluorescence intensity plot of the TK6 microcolony in the middle image after background correction (e.g., binary mask using Otsu thresholding method). The F/M is calculated as the total area under the curve.

FIGS. 9A-9C. FIG. 9A. Exemplary calculation of integrated fluorescence intensity per microcolony (F/M) for microwells with one cell (left) and five cells (right). Top: fluorescent images of TK6 cells in microwells stained with Vybrant® DyeCycle™ Green. Bottom: Fluorescence intensity plots of the corresponding fluorescent images after background correction (binary mask using Otsu thresholding method). F/M is the total area under the curve. FIG. 9B. Average F/M values for 1 to 7 TK6 cells. F/M for a single cell, or fluorescence intensity per cell (F/C), is calculated to be 2300±1500 (arbitrary fluorescence unit). Each data point is an average of 3 independent experiments. Error bars are standard errors of the means. FIG. 9C. Fold change of median F/M for TK6 microcolonies in μCC (Medium F/M) and TK6 cell density in liquid culture (Cells/mL) during 4 days of culture. Each data point is an average of at least 3 independent experiments. Error bars are standard errors of the means.

FIG. 10. Example of TK6 microcolony size distributions obtained from μCC during 4 days in culture. F/M values of >700 microcolonies were analyzed for each distribution and converted to cell numbers by dividing by the value of 1 F/C. y-axis for each plot is individually scaled.

FIG. 11. An illustration of an F/M distribution of microcolonies. i is the F/M bin number (i>0). The width of each bin is 10³ (1 F/C). F/M(i) is equal to i×10³. f(i) is the relative frequency of microcolonies in the i^{th} bin, which have F/M values between (i−1)×10³ and i×10³. Σf(i) equals to 100%. Example: f(4)=9% means 9% of microcolonies have F/M values between 3×10³ and 4×16³ and have approximate 4 cells each.

FIGS. 12A-12C. New metric for growth assay: Proliferating Fraction (PF). FIG. 12A. Proliferation fraction is calculated by subtracting the control population and quantifying the population percentage that consists of colonies having sizes greater than the median size of the control population. FIG. 12B. Survival curve using PF. FIG. 12C. Published TK6 survival curve after BCNU treatment.

FIGS. 13A-C. Illustration of proliferating fraction (PF) calculation. FIG. 13A. Notations of terms used to describe F/M distributions and definition of terms used for PF calculation. FIG. 13B. Superimposition of F/M distributions for starting population (P₀) and the population after t days on ToxChip (Pₜ) (illustrative data). Example calculations of Δf(3), Δf(4), and PF(4). FIG. 13C. F/M distribution of the subtracted population (P₁-P₀). Example calculation of total PF (illustrative data).

FIGS. 14A-14B. Example of F/M distributions of TK6 microcolonies following γ-radiation treatment. FIG. 14A. (Left) F/M distribution of starting TK6 population, which is TK6 microcolonies after 1 day on ToxChip. (Middle) F/M distribution of TK6 microcolonies after 3 more days on ToxChip and no exposure to γ-radiation, (Right) F/M distribution of TK6 microcolonies on ToxChip 3 days after γ-radiation treatment. FIG. 14B. F/M distributions for “Untreated” and “2 Gy” from FIG. 14A after subtracting the starting population (left plot in FIG. 14A). PF values are calculated according to method shown in FIG. 13.

FIGS. 15A-15B. FIG. 15A. Definition of total PF fluorescence (PF_{F}). FIG. 15B. Calculation examples of PF_{F} for TK6 F/M distributions of “Untreated” and “10 μM BCNU” after subtracting a common starting population (see FIG. 13 for an illustration of the subtraction method).

FIGS. 15C-15D. Illustration of Excess Growth calculation. FIG. 15C. Illustrative example of microcolony size distributions before (starting population in light green) and after growth (final population in dark green). Excess Microcolonies (non-overlapping dark green) are microcolonies in the final population that have grown beyond the starting population in size. Excess Growth is defined as the total number of cells in Excess Microcolonies. FIG. 15D. Simplified illustrative size distributions for 100 starting microcolonies (light green) and 100 final microcolonies (dark green) with step-by-step calculations for Excess Microcolonies and Excess Growth.

FIG. 16. Distribution of F/M among, microcolonies grown under control conditions, or challenged by exposure to BCNU. BCNU inhibits growth of colonies.

FIGS. 17A-17G. Survival curves of TK6 cells after γ-ray exposure. FIG. 17A. Comparison of TK6 survival curves after γ-ray exposure obtained from XTT, arid ToxChip assays. After γ-ray exposure, TK6 cells were cultured for 3 days for XTT, and ToxChip assays and 21 days for the clonogenic assay. FIGS. 17B and 17D. CTG® analyses of γ-irradiated TK6 cells with different plating densities 3 and 5 days post-treatment, respectively. FIG. 17C and 17E. Comparison of survival curves from clonogenic, PF_{F}, and CTG® analyses of 3.75 K cells/mL plating density 3 and 5 days post-treatment. FIG. 17F. RealTime-Glo™ MT analyses for TK6 cells with different plating densities 3 days post-treatment. FIG. 17G. Comparison of survival curves from clonogenic, PF_{F}, and the average results of all plating densities for RealTime-Glo™ MT.

FIGS. 18A-18B. Application of the PF_{F} method to study the role of DNA repair in cell survival. FIG. 18A. TK6 and TK6+MGMT cells show similar sensitivity to γ-ray exposure. FIG. 18B. MGMT significantly rescues TK6 cells from BCNU's toxicity. Error bars are SEMs of 3 or more independent experiments.

FIGS. 19A-19B. Application of the PF_{F} method to study the role of xenobiotic metabolisms in cell survival. Cells on ToxChip were treated with AFB₁ for 24 hours and recovered for 3 days in fresh culture. FIG. 19A. MCL-5 cells are significantly more sensitive to AFB₁ than TK6 cells. FIG. 19B. Co-treatment with ketaconozole (KET, a strong inhibitor of p450s that give rise to AFB1 metabolism) significantly rescues MCL-5's sensitivity to AFB₁. Error bars are SEMs of 3 or more independent experiments.

FIGS. 20A-20B. Multiplexing capacity of ToxChip. FIG. 20A. Live/DEAD staining of TK6 microcolonies on ToxChip. Live cells are positive for Calcein-AM (left panels), and dead cells are positive for EthD-1 (right panels). FIG. 20B. Annexin V staining for apoptosis. Dead cells are positive for both Annexin V-Alexa 488 (left panels, false coloring) and EthD-1 (right panels).

FIG. 21. Example bright-field pictures of wells with TK6 cells (untreated or γ-irradiated with 4 Gy) cultured in U-bottom 96-well plates over 18 days (D0=immediately after exposure, D9=9 days after exposure, D14=14 days after, and D18=18 days after) (see Methods). Pictures are taken from the same 24 wells for each condition. Obvious colonies appear at different times across the wells. The circled wells represent examples of colonies are not obvious until day 18 (D18).

FIGS. 22A-22F. Comparison of μCC with other assays in measuring γIR-induced toxicity in TK6 cells. Toxicity is expressed as percent of γ-irradiated cells relative to untreated control cells. μCC data are normalized excess growth values obtained from TK6 microcolony size distribution analysis 3-4 days after γ-irradiation. FIG. 22A. The colony formation assay data (see Methods) were obtained from TK6 cells 3 weeks after γ-irradiation. FIG. 22B. Colony formation data from *Wenz F. et al, 1998 were reproduced with permission from Radiation Research journal. FIG. 22C. XTT data were obtained 3 days after exposure (see Methods). FIG. 22D. CellTiter-Glo® (CTG®) data are for TK6 seeding density of 400 cells/96-well and 4-day recovery period. FIG. 22E. γIR-induced toxicity in TK6 cells measured by CTG® with different cell seeding densities (legend: number of cells per 96-well) and 2 different recover); periods (left: 3 days; right: 4 days). FIG. 22F. γIR-induced toxicity in TK6 cells measured by μCC with different cell loading densities (legend: number of cells per macrowell) and 2 different recovery periods (left: 3 days; right: 4 days). n≥3, error bars are standard errors of the means.

FIGS. 23A-23D. μCC analyses to measure toxicity presented as percent of treated cells relative to untreated control cells (% Control). FIG. 23A. N,N′-bis (2-chloroethyl)-N-nitrosourea (BCNU) treatment (1 hour at 37° C.) for TK6 cells and TK6+MGMT cells. *p<0.05, Student's t-test, 2-tailed, unequal variance. FIG. 23B. γ radiation treatment for TK6 cells and TK6+MGMT cells FIG. 23C. Aflatoxin B₁ (AFB₁) exposure (24 hours at 37° C.) for TK6 cells and MCL-5 cells. *p<0.05, Student's t-test, 2-tailed, unequal variance. FIG. 23D. Parallel treatment of MCL-5 cells with AFB₁ or AFB₁ in conjunction with ketoconazole (KET). *p<0.05, Student's t-test, 2-tailed, paired. All data points are means of ≥3 independent experiments. Error bars are standard errors of the means.

### DETAILED DESCRIPTION OF INVENTION

Provided herein are devices and methods useful in the study of cell maintenance, growth, and proliferation in vitro. These devices and methods are particularly useful in the analysis and potential identification of growth-modifying agents that stimulate or inhibit cellular proliferation including for example cytotoxic agents.

This disclosure provides a device upon which cells are plated and cultured under appropriate conditions and for appropriate times. The device is interchangeably referred to herein as a ToxChip and a microcolony chip (μCC).

Briefly, the ToxChip (or μCC) comprises a plurality of microwells formed in a matrix (e.g., a semi-solid matrix such as an agarose matrix) and optionally one or more physical barriers that divide and separate each plurality of microwells from other pluralities of microwells. The physical barriers may create a macrowell within which a plurality of microwells (e.g., less than 50 to 200, or more) are situated. Each plurality of microwells may be treated in a unique manner (e.g., one may be an untreated control, another may be treated with a first agent, and optionally another may be treated with a second agent, etc.).

Cells are seeded within each microwell, and the number of cells between microwells may vary from zero to the maximum number of cells that can be physically located within the microwell. Thus, the number of cells that can be seeded per microwell at the beginning of a culture will be controlled in large measure by the size (dimensions) of the microwells. This will be the case regardless of the cell density of the cell suspension added to the microwells. Importantly, the assay is not dependent on the exact number of cells initially loaded into a microwell. Cells may be exposed to an agent of interest before or during their residency in the microwells or they may be unexposed to such agent (in which case they are referred to as being untreated). Whether the cells are exposed (i.e., treated) or untreated, they are then cultured for relatively brief periods of time, after which the total number of cells per microwell is measured using total DNA content as a surrogate for total cell number. The total number of cells measured includes live and dead cells, as well as proliferating and non-proliferating cells, and is not dependent on the metabolic activity of the cell.

Significantly, the method does not require knowledge of the exact number of cells seeded into each microwell. Instead, it assumes that microwells seeded with the treated and untreated cells will have a similar seeding distribution (e.g., roughly the same proportion of microwells will be seeded with zero cells, roughly the same proportion of microwells will be seeded with 1 cell, etc. even if the number of microwells seeded with different numbers of cells in one plurality is different). The number of microwells may differ between pluralities and the total number of cells seeded may differ between pluralities. In some instances, the total number of cells seeded into a plurality of microwells in one macrowell is the roughly the same as the total number of cells seeded into another plurality of microwells in a second macrowell but the assay is equally robust even if total cell number and/or total microwell number differ between pluralities.

As described in greater detail herein, the analysis involves a determination of the colony size distribution based on proportions of microwells comprising 1, 2, 3, 4, 5, 6, etc. numbers of cells (i.e., microcolonies having 1, 2, 3, 4, 5, 6, etc. numbers of cells), and then the proliferation fraction which again is based on microwell or microcolony proportions. As a result, it is not necessary that the same number of cells be seeded in each microwell. In this way, the current method and assay are further distinguished from existing assays which require that the same seeding cell number (e.g., XTT/MTT and CTG).

The readouts from such an assay include the colony size distribution profile of treated cells (particularly in comparison to the same profile in untreated cells), a measurement of the proliferation fraction of the treated cells (also referred to herein as “excess microcolonies”), and a measurement of the total fluorescence intensity of the relative proliferation fraction (also referred to herein as “excess growth”). These readouts have been shown to be as sensitive as clonogenic assays, which heretofore have been considered the most sensitive assays available for measuring proliferation and toxicity. Surprisingly, the method is far less time and labor intensive than clonogenic assays or other less sensitive and more costly assays.

This disclosure refers to the cells present in a microwell after a period of culture as a microcolony. However, as explained in greater detail below, this does not intend that the population is monoclonal. The assay simply measures the total number of cells in the microwell after culture (e.g., the microcolony) regardless of whether the cells are progeny of one cell or multiple cells. The assay is also not dependent on the ability to observe and count demarcated, physically separate colonies in single microwells.

As used herein, the term “microcolony” refers to a one or more cells present in the same microwell that may or may not be clonal (i.e., they may or may not have derived from the same initially seeded cell). Cells in the microcolony may infiltrate the matrix. In order to calculate F/M and PF, it may be necessary to define the minimum surface area occupied by a microcolony. In some instances, the microcolony may be defined as having a minimum surface area of 50 um² (i.e., the area occupied by a single cell).

A more detailed description of the assay is provided below.

Thus, the ToxChip is an assay for cell growth, including growth inhibition, that can be used for virtually any cell type that can be cultured in vitro including but not limited to mammalian cells such as human cells and prokaryotic cells such as bacterial cells. The principle is to seed live cells into a microarray of micron scale wells, provide growth media and culture conditions for various amounts of time, and finally to measure the total number of cells following such culture. Healthy cells double approximately once every 24 hours on average, thus within only a few days one can detect significant increases in colony sizes. This is a significant improvement over standard clonogenic assays that require far longer periods of time, particularly when they are dependent on visualizing colonies by eye.

The total number of cells per microwell (referred to as a microcolony, regardless of whether the cells are monoclonal) is estimated by measuring the total DNA per microwell. To achieve these measurements, colonies are stained for DNA and subsequently imaged. Image processing gives the integrated fluorescence intensity per microcolony. As an example, approximately 50 colonies may be processed in a single image.

For each treatment condition, a few to up to thousands of microcolonies are queried in an automated fashion, giving rise to the distribution of colony sizes. The size distribution reflects growth of these colonies. There is no limit to the number of microcolonies that may be queried for any given condition. Physical barriers can be used to separate pluralities of different conditions. As described herein, one non-limiting example of such physical barriers are bottomless wells in a 96 well plate. Thus, to illustrate, each well of the 96 well plate is referred to as a macrowell and such macrowell represents a treatment condition. Within each macrowell, there may be tens to hundreds of microwells, depending on the size of the macrowell and the size of the microwell. If the macrowell is defined by a well in a 96 well plate and the microwells have an average diameter of 40 microns in both diameter and depth, then typically there will be about 200 microcolonies/macrowell. It is to be understood however that the number of microcolonies per macrowell may be different and are not limited to simply the numbers provided herein. It is also to be understood that multiple macrowells can have the same condition and their microwells can be queried together. It is also to be understood that any type of partition may be used provided it is capable of physically penetrating the matrix thereby created isolated (physically separate) regions (and thus pluralities) of microwells. Any given analysis for a particular treated or untreated condition may involve anywhere from hundreds of microwells (e.g., 100, 200, 300, 400, 500, 600, 700 or more) to thousands of microwells (e.g., 1000, 2000., 3000, 4000, 5000 or more), and thus may involve single macrowells or pluralities of macrowells.

It is also to be understood that the number of cells which may be initially loaded into each microwell will depend upon the size of the microwell and the size of the cells. In the exemplary, non-limiting embodiments described herein, typically a range of 0-7 cells are loaded into the microwells initially. However the assay is not so limited, and more cells may be initially seeded including for example tens, hundreds, or thousands of cells may be loaded per microwell.

In some instances, after loading cells into the matrix, cells can be exposed to different conditions. To learn about the impact of those conditions, samples are removed from the tissue culture incubator (if analysis is for eukaryotic cells) at various times (days 1, 2, 3, and 4 for example). Cells that are unexposed (untreated) typically divide at regular intervals (e.g., roughly 24 hours for mammalian cells). In contrast, cells exposed to an agent that inhibits cell division or that is toxic (treated) give rise to smaller colonies (or non-existent colonies, depending on the level of toxicity).

Conditions can be selected that are toxic. For example, cells can be exposed to a known DNA damaging agent (possibly a test agent). Dead or dying cells will not proliferate and thus will not contribute to the increase in size of a microcolony. As a result, healthy cells form a broad range of colony sizes, whereas treated cells form very small colonies because most if not all cells die or cannot divide (mitotically arrested). By subtracting the starting control (untreated) colony size distribution from the test (treated) colony size distribution, one can measure the relative ability of cells to form microcolonies following exposure to the agent of interest. Using this approach, a dynamic range of up to three orders of magnitude can he obtained.

The assay is substantially more sensitive than the most common toxicity testing assays that rely on dyes that are sensitive to mitochondrial activity. Although such assays (e.g., MTT, XTT and CellTitre-Glo) are routinely used in high throughput screens, these assays afford a very narrow dynamic range of approximately one order of magnitude (in the case of XTT and MTT) and their results are commonly subject to artefacts such as those due to interactions between the dye and components of the media or due to biological responses of cells that are not related to cell survival. The ToxChip, on the other hand, overcomes these limitations because it measures cell numbers via total DNA content (i.e., it comprises growing cells on the chip, applying a standard DNA stain to such cultured cells, and imaging the cells to measure the total stain intensity). Additionally, the ToxChip requires less sample handling.

The ToxChip approach gives rise to results that are highly comparable to what can be observed using the traditional clonogenic assay. However, unlike the clonogenic assay, the ToxChip takes days instead of weeks, is equally sensitive, requires far less reagents, and is less labor intensive. ToxChip is also compatible with high throughput screening equipment. Data collection minimally requires an epifluorescent microscope at low power. It is to be understood that the type of imaging modality used will be dictated by the stain applied to the cells to measure the DNA content. The cells may be stained with luminescent or radioactive stains or labels and corresponding imaging techniques would be used to detect signal.

The ToxChip can be used in a number of applications and may be varied relative to the standard exemplary disclosures provided herein. For example, it can be used to monitor cell cycle arrest. It can be used at a single cell level to perform identically to a clonogenic assay. It can be modified to culture virtually any cell type of interest by modifying the semi-solid matrix and/or the substrate, and/or the overlay. Additionally, analysis of the colony size distribution can be used as an indicator of cell to cell variability. Colony morphology can also be monitored and may provide information relating to cell populations and effects of agents on such populations.

Other applications include assays that rely on formation of colonies as a readout. For example, anchorage-independent growth is one of the hallmarks for neoplastic transformation. Typical cell transformation assays plate anchorage-dependent cells in soft agar and count the number of colonies after a couple of weeks as a measure of the number of cells that have gone through neoplastic transformation. ToxChip is inherently based on agarose and therefore can be used to screen for occurrence of anchorage-independent proliferation of cells that normally require attachment to external ligands.

Another type of colony-based assay is the mutation assay, such as the HPRT and MLA assays. The principle is to treat cells with a mutagen and grow cells in a special culture condition where only the mutants can survive and form colonies. The frequency of colonies will therefore represent the frequency of mutations. We can use ToxChip to calculate the number of cells that can grow in the special culture condition (which then yields information about the relative frequency of mutants).

### ToxChip Preparation

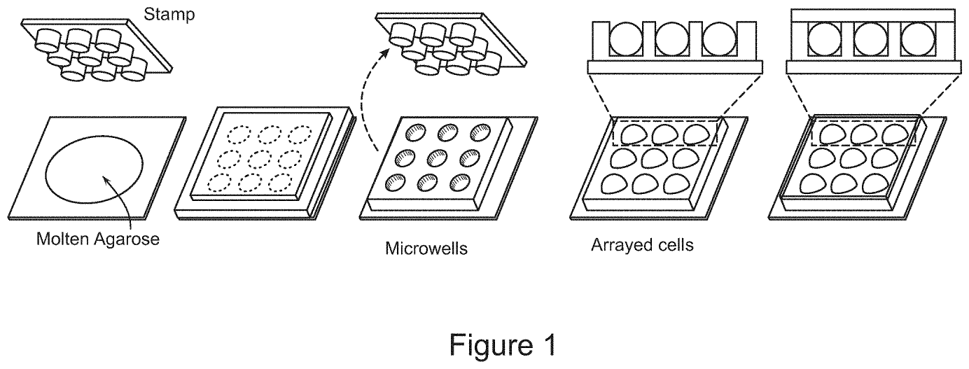

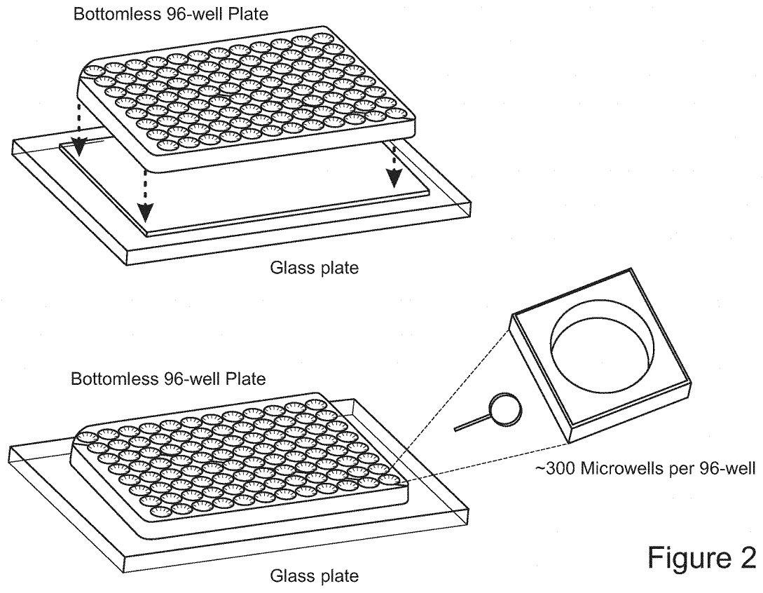

The ToxChip comprises a number of microwells microfabricated in a matrix. Virtually any type of biologically compatible polymer can be used as the matrix. The matrix may be semi-solid or solid, depending on the application. Examples of semi-solid matrices include but are not limited to agarose. Other suitable matrices include other types of hydrogels alginate) and polydimethylsiloxane. Solid materials could include tissue culture treated plastics. The microwells are typically arranged in a fixed array, as illustrated in FIG. 1. Cells are loaded by gravity and a matrix is then overlayed on the cells. If the cells are adherent cells, they may be additionally overlayed with one or more extracellular components such as but not limited to collagen, fibronectin, gelatin, etc. The matrix overlay is intended to retain cell (and progeny) position during the assay. FIG. 2 illustrates the positioning of an example of a physical barrier (in the form of a bottomless 96 well plate) onto the fixed array of microwells. The 300 microwells per macrowell is non-limiting. Micropatterning of cells and automated image analysis significantly increase the throughput and sensitivity of the assay (33)

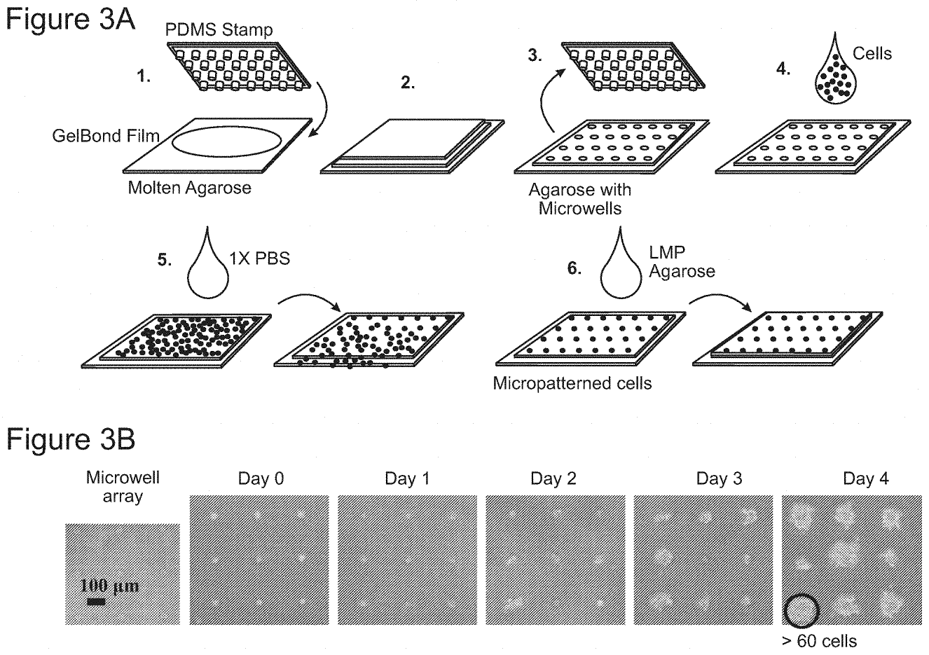

Arraying cells in a micropattern makes it possible to measure colony formation using a small area. Specifically, the microarray increases the density of colonies per cm² by ˜250 times while eliminating most of the microcolony overlap. In one embodiment, cells may be arrayed as follows: A microfabricated PDMS mold (e.g., created by soft photolithography) is pressed into molten agarose (e.g., 1% normal melting point (NMP) agarose in complete culture medium). The agarose is allowed to gel, and the mold is removed to reveal an array of microwells. For the experiments described here, each microwell is ˜40 μm in both diameter and depth, spaced ˜240 μm apart from one another. The microwell array platform provides a tunable physical distance between microcolonies and tunable well sizes. For example, microwell sizes can be as small as 10-20 μm in diameter and as large as is desired. Distance between wells is fully scalable. A bottomless 96 well plate is then compressed on top of the microwell array to create macrowells with more than 200 microwells each. A solution of cells is then placed on the microwell array and the cells are loaded into the microwells by gravity. Excess cells may be removed by washing or by sheer force. Upon removal of the excess cells, a microarray of cells is revealed (FIG. 3A). After settling into the wells by gravity, cells are trapped by adding for example low melting point (LMP) agarose (e.g., 0.25%, typically in complete culture medium) in a layer above the cells. The chip is then submerged in complete culture medium, and optionally such medium is changed in whole or in part regularly (e.g., every day, every two days, etc.).

Using the microwell array, we have shown that the TK6 human lymphoblast cell line can be micropatterned (FIG. 3B). After the cells were loaded into the microwells, we observed an average of approximately three TK6 cells per microwell (FIG. 3B—Day 0). We then demonstrated that TK6 cells incubated in cell culture media at 37° C. in these microwells were able to grow. Appearance of cells growing out of the microwell boundary was noted as soon as two days in culture (FIG. 3B, Day 2). By day four, most cells in microwells had formed large microcolonies (FIG. 3B, Day 4). Thus, any cell type that grows in a solution without a growth surface can be analyzed directly on the simple matrix (e.g., agarose) version of the ToxChip.

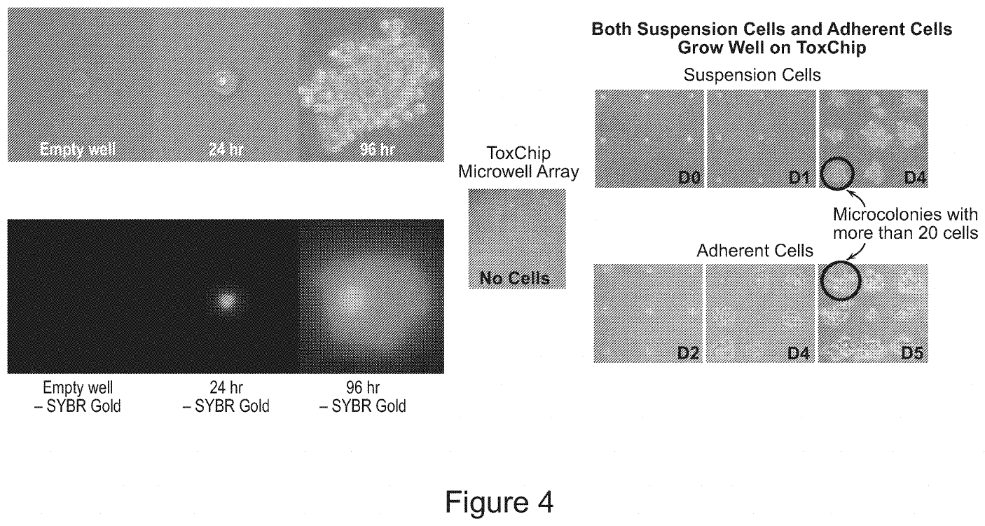

We further demonstrated that adherent cells could also be cultured using the microwell array. HeLa, human cervical carcinoma epithelial cell line (adherent), considered representative of adherent cells, was grown using an overlay of collagen (e.g., Type I collagen gel) situated between the cells and the LMP matrix. The HeLa cells attached to the collagen overlay and thus grew upside down in the encapsulated microwell. The growth of both the non-adherent TK6 cells and the non-adherent HeLa cells is shown in FIG. 4. The assay can be modified to overlay other extracellular matrix components and/or ligands on the cells.

The cells may be grown for any period of time, including for example 1, 2, 3, 4, 5, 6, or 7 days. Robust results can be obtained using cells grown for about 1-4 days.

### Staining of Cells and Microcolonies

Following culture, the cells may be exposed to a DNA-specific dye such as a DNA-specific fluorescent dye. A DNA-specific dye is one that preferentially and potentially exclusively binds to DNA and not RNA or other moiety in the cell. In some instances, the cells may be exposed to a nucleic acid specific dye (i.e., one that binds to DNA and RNA) provided RNA levels are relatively consistent among the cells. In some instances such dye is also membrane-permeable and thus the cells do not need to be lysed for the dye to enter the cells. Thus, the cells need not be treated with a lysing agent such as a detergent prior to staining. Examples of membrane-permeable DNA-specific fluorescent dyes include but are not limited to Vybrant® Dye Cycle™ stains including Dye Cycle™ violet, Dye Cycle™ green, Dye Cycle™ orange, and Dye Cycle™ ruby, the family of Cyanine stains including (Blue-Fluorescent SYTO, Green-Fluorescent SYTO, Orange-Fluorescent SYTO, and Red-Fluorescent SYTO, Hoechst, Acridine orange. NUCLEAR-ID® Red DNA stain, and NUCLEAR-ID® Blue DNA stain. If a membrane-impermeable dye is used, then the chip may be embedded in a cold detergent comprising buffered solution in order to solubilize the membrane. Examples of membrane-impermeable dyes include SYBR gold, DAPI and PI. Membrane-permeable dyes are preferred.

Other stains or probes may he used to quantitate DNA content including luminescently and radioactively labeled probes or stains.

Once the cells (or microcolonies) are stained, excess dye is removed and the microcolonies are imaged. Images may be acquired using an epifluorescent microscope. imaging can be done automatically using an automated stage such as a motorized scanning stage, or other high throughput imaging platform. The entire analysis for imaging and image processing takes minutes to complete.

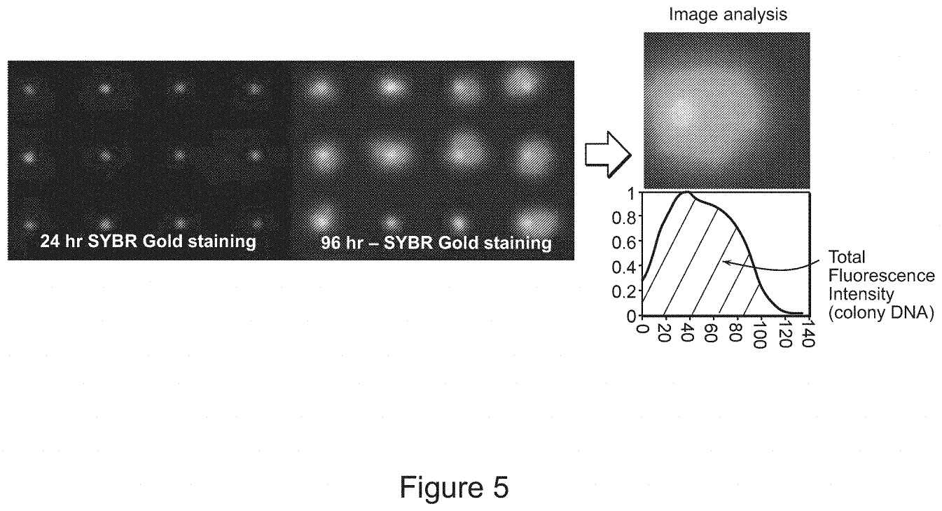

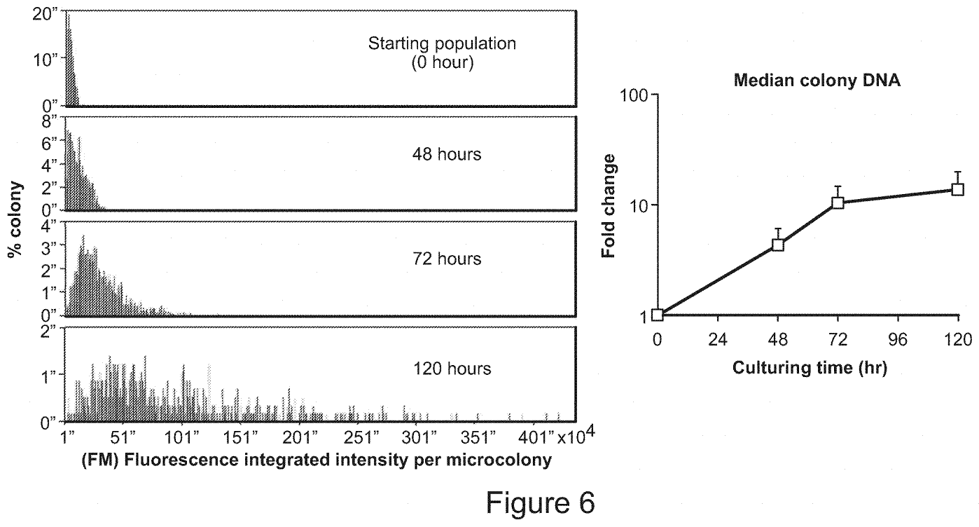

A program was developed to measure total integrated fluorescence intensity per microcolony (referred to herein as F/M). Total integrated fluorescence intensity per microcolony is proportional to total DNA per microcolony. Total DNA fluorescence intensity of each colony was quantified using this program, as illustrated in FIG. 5. FIG. 6 further shows data for TK6 colony formation that was monitored over 120 hours after cell seeding. A clear shift of total DNA intensity from left (small colonies) to right (large colonies) over time is observed.

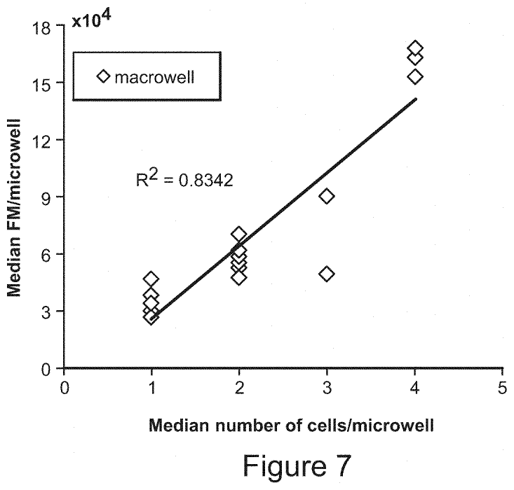

The amount of DNA in a colony, such as a microcolony, correlates with the number of cells in the colony. In order to test the relationship between F/M and number of cells per microwell, the median number of cells per microwell and the median F/M per micro well for eighteen macrowells were compared. The number of cells for each microwell was counted by morphology using an phase-contrast microscope. The number of cells was recorded for approximately fifty microwells per macrowell and the median value was calculated for each macrowell. These cells were then stained with SYBR Gold and imaged using an epifluorescence microscope. For each macrowell, between 23 and 112 microwells were imaged and analyzed for their F/M values. The median F/M for each macrowell was calculated and plotted against the median number of cells. A strong linear relationship was observed between them (R²=0.8342; FIG. 7).

### High-Throughput Quantification of Microcolony Size Via Nucleic Acid Fluorescence Staining

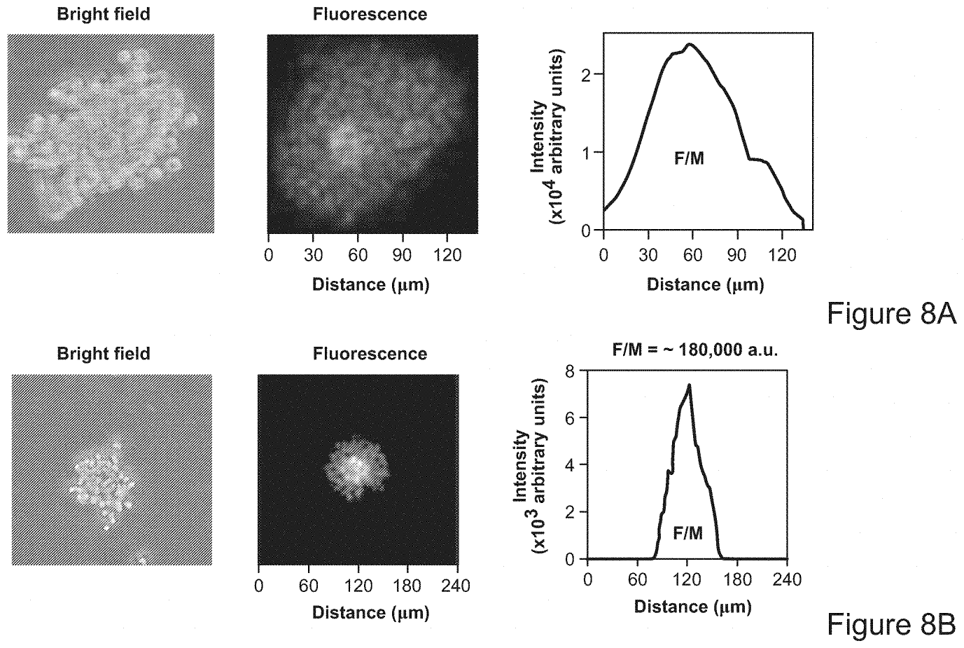

In order to quantify the size of the arrayed microcolonies, individual microcolony sizes were estimated based on the total DNA content for each microcolony. DNA content is a useful indicator of cell number. The DNA of the microcolonies was labeled using a DNA-specific fluorescent stain, such as Vybrant® DyeCycle Green. Fluorescent images of microcolonies were then captured (FIGS. 8A-8B). To quantify total DNA staining for each microcolony, a program that integrates the fluorescence intensity for a given area was developed. Briefly, a fluorescent image of arrayed microcolonies is input into the program. The program then detects the locations of all the microcolonies in this input image and generates images of individual microcolonies (example in FIG. 8A, middle). To define the area for each microcolony, the boundary of each micro-colony was set to be one half the distance between two microwells (in this instance, ˜120 μm). For each image of a microcolony, the program generates a plot of the average fluorescence intensity of each pixel column from the left to the right of the image. See FIGS. 8A and 8B, right plots. To attain the integrated intensity of a colony, an intensity scan was generated and the total fluorescence intensity was derived from the total area under the curve as in FIGS. 8A-8B (right plots). The total fluorescence per microcolony was defined as F/M (or F/M, as the terms are used interchangeably herein). FIG. 8B shows a similar analysis except that the data in FIG. 8B (right plot) has had a background correction factor applied to it.

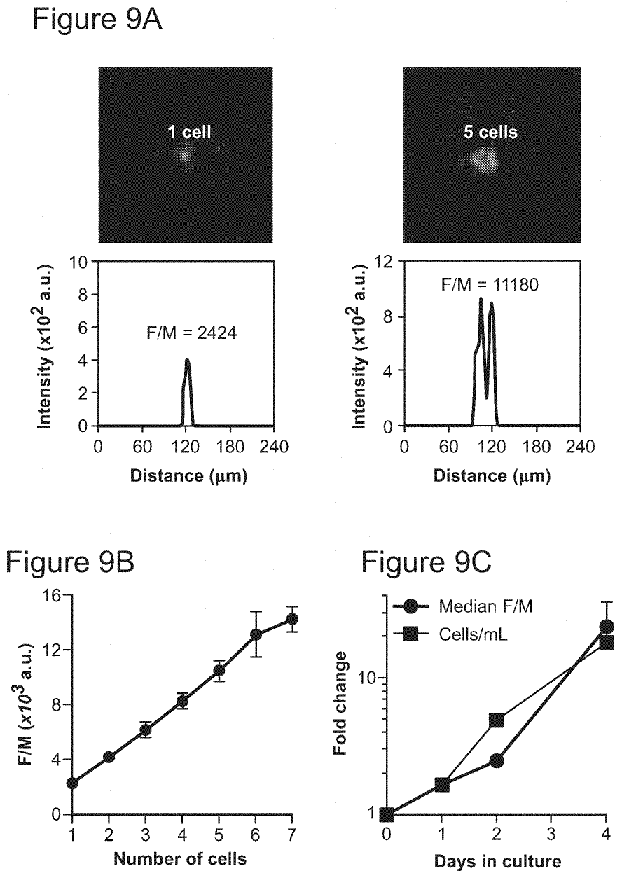

To study microcolony sizes via F/M, first it was determined whether F/M is a reliable measurement of cell numbers in μCC. TK6 cells were loaded into an array of 40-μm wells and immediately stained the cells with Vybrant® DyeCycle Green. The microwells provide physical spaces with a defined volume that only allows a maximum number of ˜7-8 TK6 cells per well. Fluorescent images of TK6 microcolonies were captured and analyzed using an in-house MATLAB program as described above. Because the cells had not been given time to grow on the μCC, the fluorescent nucleus of each cell in a microwell can be clearly distinguished by eye in the fluorescent images. The number of distinct fluorescent nuclei in a microwell was counted by eye as an estimate of total cell number for that microwell and was compared against the micro well's F/M value (examples in FIG. 9A). As shown in FIG. 9B, the number of cells per microcolony increases linearly with the microcolony's F/M value (R²=1), indicating F/M is a sensitive and robust measurement of cell number up to 7 cells. F/M for a single cell, or fluorescence intensity per cell (F/C), is calculated to be 2300±500 (arbitrary fluorescence unit).

To further investigate whether F/M is a suitable measurement of microcolony size beyond the cell number countable by eye (7 cells), the change in the median F/M value was monitored over 4 days in culture. Regression analysis shows that the median F/M for TK6 microcolonies increased exponentially between day 0 and day 4 on the μCC (R²=0.95) with a doubling time of approximately 21 hours. This is similar to the exponential growth of TK6 cells in liquid culture where the fold change in cell density (number of cells/mL, determined by an automated Vi-CELL™ cell counter from Beckman Coulter Life Sciences) also doubles every ˜21 hours (FIG. 9C). It was concluded that F/M is a sensitive and robust measurement of microcolony size and that the environment of the μCC does not significantly affect the growth rate of TK6 cells.

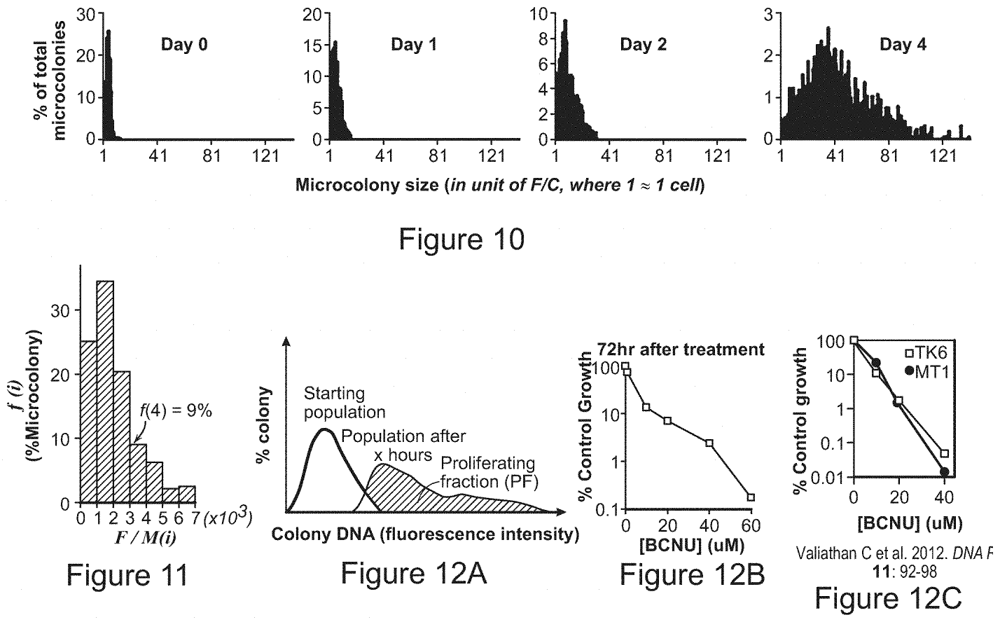

The growth of the TK6 microcolonies over the course of four days was monitored. Initially, the number of cells per microwell ranged between 1 and 7 cells. On each day, a plate was removed for analysis and microcolonies were stained for DNA content. The F/M values were quantified and an F/M distribution for TK6 microcolonies was generated for each day in culture (FIG. 10). As expected, some microcolonies remained very small, while others had grown extensively. By day 4, microcolony F/M values ranged from 1 F/C to 150 F/C, corresponding to ˜1 cell up to ˜150. It is important to note that a colony that started off with 7 cells could readily double to form a colony of more than 150 cells over the course of four days (approximately 4.5 doubling times). FIG. 10 shows that the F/M distribution of TK6 microcolonies is very tight on day 0 and that as the microcolonies grow, the distribution both shifts to the right and broadens. The extent to which the populations become broader when all plots have the same scale for the y-axis is readily apparent (data not shown). It was postulated that the broadening of F/M distributions is attributable to the difference in starting microcolony sizes, growth rates, and potential effects of cell-cell interactions.

Construction of Microcolony Size Distribution using F/M Values

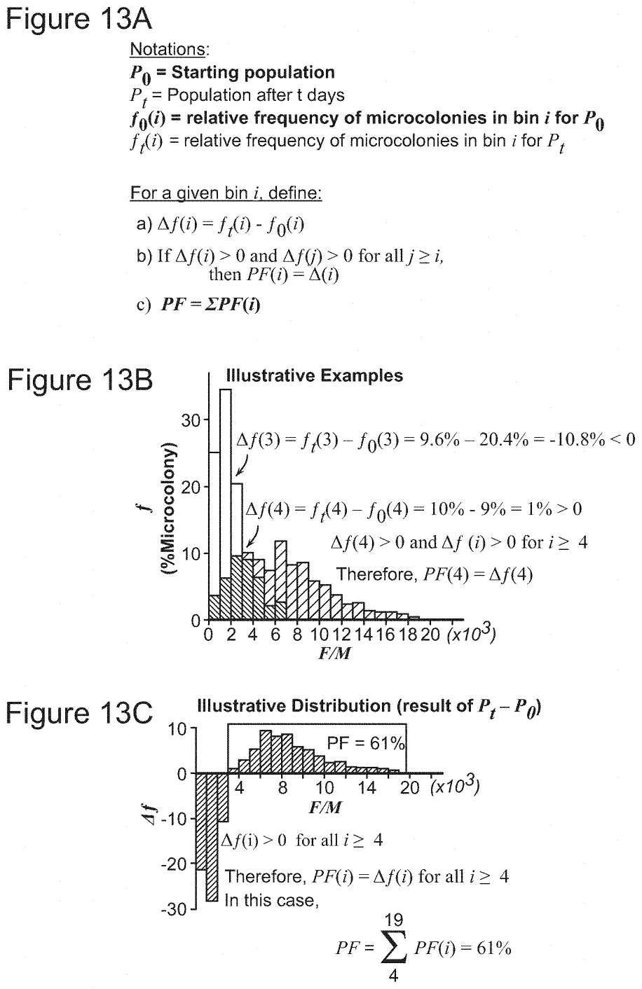

In order to study the distribution of microcolony sizes, first the fluorescence intensity per cell was defined. F/M data was collected for single cells and then averaged to estimate the fluorescence intensity per cell (1 F/C=˜1×10³). The F/M distribution of microcolonies was derived by calculating the frequency of microcolonies with F/M values that fall within an F/M bin. FIG. 11 illustrates an F/M distribution of microcolonies on ToxChip. The width of an F/M bin was chosen to be one F/C, which means the microcolonies that fall in the same F/M bin have approximately the same number of cells. i was defined to be the F/M bin number and f(i) to be the relative frequency of microcolonies in the i^{th} bin. Therefore, Σf(i)=100%.

### Proliferation Fraction (PF)

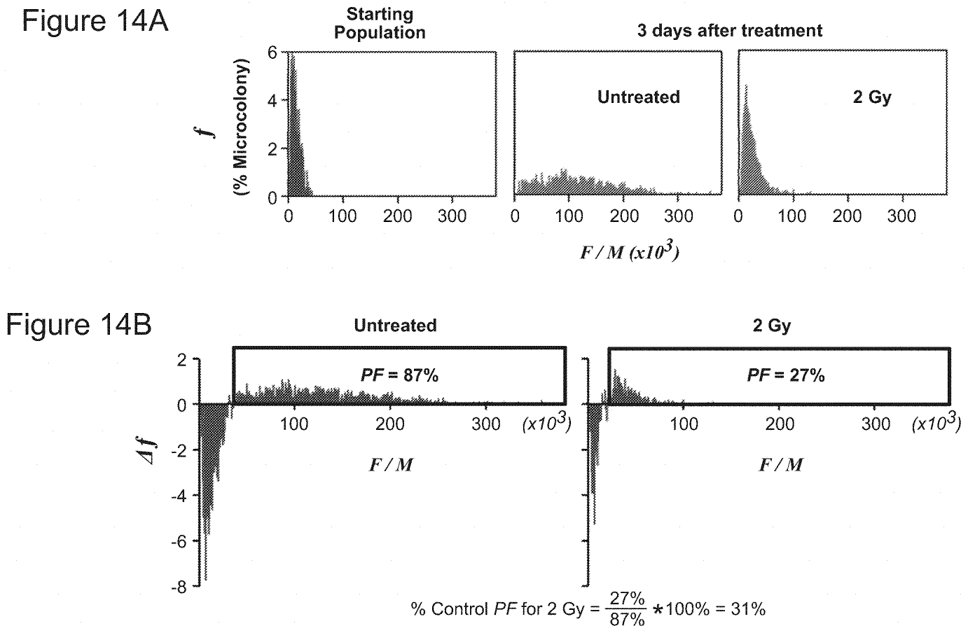

This disclosure defines a new parameter, proliferating fraction (PF). Briefly, in a given population distribution at time x, PF is the population fraction that has shifted to the right of the starting population distribution. In other words, a proliferating fraction of a population approximates the percentage of colonies that have increased in size at time x (FIG. 12A). An exemplary method for calculating PF is provided below. When this measure is used, an exponential toxicity curve (FIG. 12B) was obtained that spans three orders of magnitude of detection and resembles published results using a colony forming assay (FIG. 12C) (5).

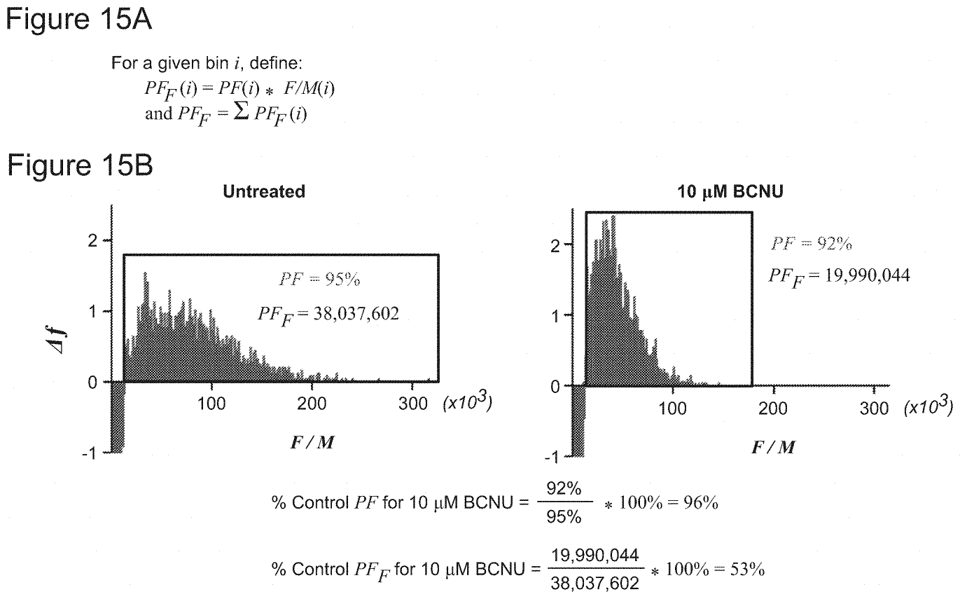

To estimate toxicity, the change in F/M distribution after a toxic treatment was quantified and compared to the change in F/M distribution of the untreated population. Several parameters to use as measures of the change in F/M distributions were defined and compared: median F/M, proliferating fraction (PF), and total fluorescence of the proliferating fraction (PE_{F}). The PF of a population was defined to be the total increase in percentage of larger microcolonies compared to the starting population. FIG. 13B illustrates how the calculation is conducted. The starting population (P₀) is subtracted from the population after t days in culture (Pₜ), and the change in relative frequency of different microcolony sizes is examined (Δf(i)=fₜ(i)−f₀(i) for bin i). If Δf(i) is positive and Δf(j) is also positive for all bins j>i, then PF(i) was defined to be Δf(i). The total proliferating fraction (PF) was calculated by a summation of all PF(i). FIG. 13C shows the F/M distribution for Δf to further demonstrate the calculation method. Since Δf(i)>0 for all bins starting from i=4, PF(i)=Δf(i) for all i≥4. There are 19 bins in total. Therefore, PF=Σ₄¹⁹PF(i)=61%

To demonstrate the utility of PF in toxicity measurement, micropatterned TK6 cells were treated with the highly cytotoxic γ-radiation. Exposure to γ-rays directly causes DNA single strand breaks and double strand breaks, which can be highly toxic. Studies using the colony formation assay have shown that TK6 cells are very sensitive to γ-radiation (1-4).

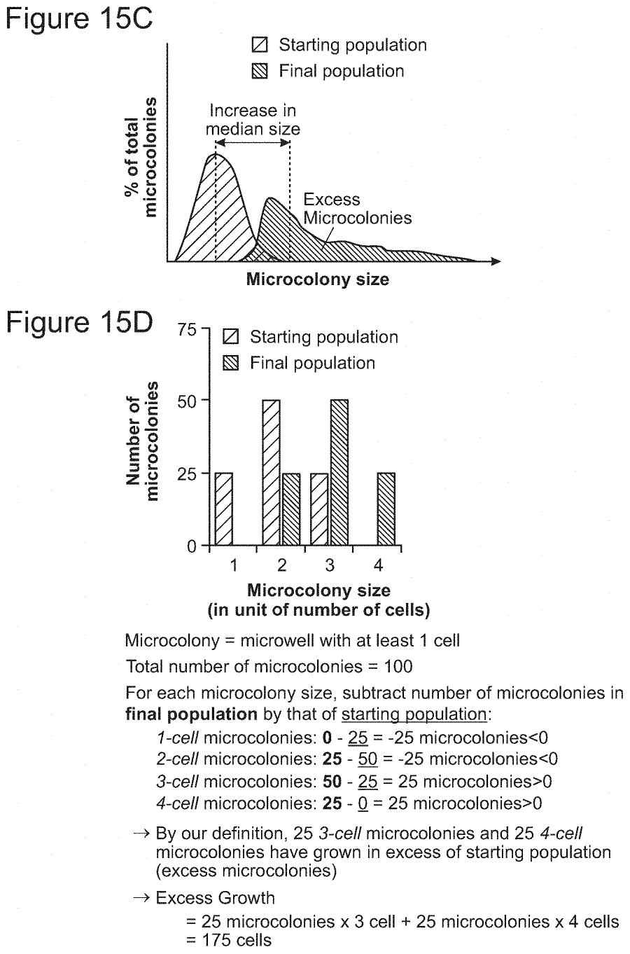

TK6 cells were treated with various doses of γ-radiation and monitored the distribution of F/M values over three days. As expected, untreated cells readily grew into larger colonies, and they gave rise to a broad distribution of microcolony sizes (FIG. 14A—middle plot). In contrast, cells that were exposed to 2 Gy are growth inhibited either due to cell death or inability to divide, leading to reduced frequency of larger microcolonies and increased frequency of smaller microcolonies compared to the untreated F/M distribution (FIG. 14A—right plot).

FIG. 14B shows the F/M distributions for the negative control population (“Untreated”) and the “2 Gy” population after subtracting the starting population in FIG. 14A. In both distributions, negative values for Δf at lower F/M followed by positive values for Δf at higher F/M were observed (FIG. 14B). Applying the PF calculation method illustrated in FIG. 13, the PF value for the untreated sample is ˜87% and the PF for the 2 Gy treated population is ˜27% (FIG. 14B). Therefore, the PF of 2 Gy treated sample is 31% of the untreated control PF.

The terms “proliferation fraction” and “excess microcolony” are used interchangeably herein and refer to the differential gain in the proportion of microcolonies of a given cell number, at the end of the experiment, as compared to the proportion of microcolonies of the same cell number at the beginning of the experiment.

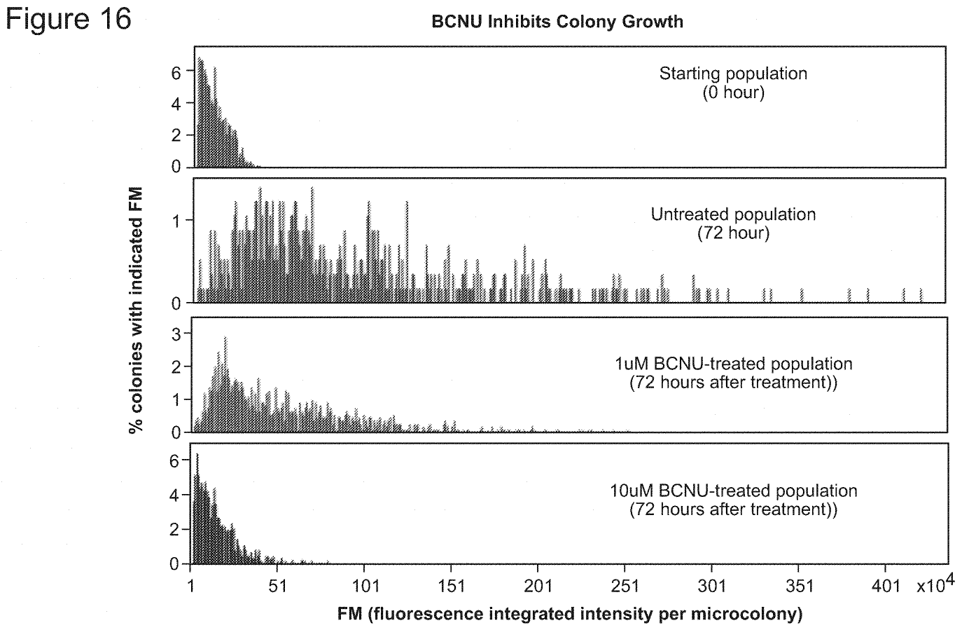

While PF captures the frequency of the growing microcolonies the calculation of PF does not incorporate information about the microcolony sizes. As shown in FIG. 15A, when TK6 cells are treated with 10 μM of the cytotoxic N,N′-bis (2-chloroethyl)-N-nitrosourea (BCNU), the PF value is 96% of the control PF, which does not indicate toxicity. However, the subtracted F/M distributions for the control and treated microcolonies show a marked difference in shape (FIG. 15B). Particularly, the PF portion of the “10 μM BCNU” has a much narrower distribution compared to the PF portion of the “Untreated”. There is also a larger frequency of microcolonies with higher F/M values in the “Untreated”. To account for the difference in the F/M values, the total PF fluorescence (PF_{F}) was defined to be the sum of total PF fluorescence for each F/M bin (notated as PF_{F}(i) for bin i) (equations in FIG. 15A). FIG. 15B shows that the PF_{F} of the 10 μM BCNU-treated TK6 cells is only 53% of the control PF_{F}, which indicates a high degree of toxicity and is more consistent with previously published studies than the results from PF (5, 6).

The terms “proliferation fraction fluorescence” and “excess growth” are used interchangeably herein and refer to an estimate of the relative number of cells (in a macrowell or in the plurality of microwells) gained in the course of the experiment as a result of cell division (and thus exclude the number of initial cells seeded in a macrowelt or in the plurality of microwells). It may be calculated by first multiplying the each excess microcolony by its cell number (per excess microcolony) and then summing such products.

Generation of Fluorescence Intensity Values from Fluorescent Images of Microcolonies

The following steps may be performed using MATLAB or other suitable programming languages. This disclosure therefore provides a program for making a computer execute the steps shown below.

The following is an exemplary set of steps that may be performed using a computer in order to generate the suitable readouts according to this disclosure.

1. The following parameters, obtained after culturing, staining and imaging cells, were input into the program:

(a) the location of fluorescent image tiles in computer. Each file contains a stack of fluorescent images of arrays of microcolonies (multiple microcolonies per image).

(b) the pixel-to-micron conversion factor of the microscope's setting (e.g., 1.61 microns/pixel for the 4× objective of a Nikon Eclipse 80i epifluorescence microscope).

(c) the minimum allowed area in an object for it to be counted as a microcolony (cell cluster). This number was set to be sufficiently low so that single-cell clusters are counted. This number is determined empirically using images of single-cell clusters. In this case, it is set to 50 μm².

(d) the physical distance between microwells (in this case, 240 μm).

2. The software reads in image files and performs the analysis on one image at a time.

3. Background correction is applied to each image to reduce all background fluorescence values to 0. Otsu's thresholding method may be used, as well as other standard methods known in the art.

4. Each image is searched for objects that are larger than the minimum allowed area (from step 3, the background correction step).

5. The locations of these objects are mapped out and the distances between them are calculated. Based on the physical distance between microwells set in step 4, objects that are not within 240 μm (or multiples of 240 μm) of any other object, are excluded from further analyses. All the remaining objects are considered “true” microcolonies and will be further analyzed.

6. The boundary of each microcolony was set to be one half the distance between two microwells (in this case, 120 μm). Accordingly, the software generated a 240 μm×240 μm image for each microcolony.

7. For each image of a microcolony generated in step 6, a plot of the average fluorescence intensity of each pixel column from the left to the right of the image is produced.

8. The area under the curve of the plot in step 7 is calculated by summing up all the average fluorescence intensity values of all the pixel columns. The result is the integrated fluorescence intensity per microcolony (F/M).

9. The output for each input file (step 1) is a text file that contains the F/M values of all the microcolonies detected in the input file.

### Determination of the Average F/M Value Per Cell (F/C):

1. Cells are loaded into microwells. For our setting, a range between 0 and 7 cells per microwell can be observed using a phase-contrast microscope.

2. These are immediately stained for DNA content with a DNA fluorescent dye.

3. Fluorescent images of cells in an array of microwells are captured.

4. The images are analyzed and the F/M value for each microwell with cells is calculated by the MATLAB software described above.

5. Because the cells had not been given time to grow, the fluorescent nucleus of each cell in a microwell can be clearly distinguished by eye in the fluorescent images. Therefore, the number of cells per microwell can be estimated by the number of fluorescent nuclei counted by eye.

6. The number of fluorescent nuclei counted per microwell ranges between 1 and 7, consistent with number of cells counted under phase-contrast microscopy,

7. The F/M values of 10 to 40 microwells with the same number of fluorescent nuclei are averaged. The results are average F/M values corresponding to 1 to 7 cells per microwell.

8. This exercise is repeated in three independent experiments.

9. The F/M values from the three independent experiments are averaged and 95% confidence intervals are calculated.

10. We define the average F/M value for microwells to be F/C. In our setting, F/C=2,300±500 arbitrary fluorescence unit (a.u.).

Generation of Microcolony Size Distribution from Fluorescence Intensity Values:

The following steps may be performed using Python programming language or other suitable programming languages.

1. The following are input into the software:
a. The location of the output text files with F/M values generated from the program as described above
b. The range of F/M values to be sorted into bins and the width of each bin. Conservatively, we set the F/M range to be between 0 and 2,000,000 a.u. to cover all the possible F/M values. Depending on the dimension of the microwells, cell types, and microcolony sizes, this range can be adjusted accordingly. The width of each bin is set to be 2,300 (a.u.), which is the value of F/C. Each bin therefore represents a microcolony size, and the bins are ordered in 1-cell increment.

2. Each F/M entry in the input text files represents a microcolony. Therefore, the total number of microcolonies in each population is the total number of F/M entries in each input text file.

3. The F/M values in each input text files are sorted into bins as specified in step 1b. The microcolonies that fall in the same bin have approximately the same number of cells.

4. The number of microcolonies, divided by the total number of microcolonies (calculated in step 2), is the relative frequency of microcolonies with the same number of cells and can be expressed as a percentage of total microcolonies.

5. The output is a worksheet (e.g., Excel worksheet) where the first column is a range of microcolony sizes between 0 and 869 cells (corresponding to the F/M range between 0 and 2,000,000 as specified in step 1b). The adjacent column contains the relative frequencies of microcolonies that have the same size corresponding to the entries in the first column

6. Multiple populations of microcolonies can be analyzed at the same time, and the results can be exported into one common worksheet

7. The relative frequencies of microcolony sizes in the output worksheet constitute the microcolony size distribution for each cell population

### Analysis of Toxicity

1. To perform a toxicity assay, cells embedded in a ToxChip are exposed to a toxic agent for a fixed time (treatment period) and allowed to recover in fresh culture for at least 3 cell divisions following treatment (recovery period).

2. The microcolony size distributions of a starting population and two final populations arc analyzed (see steps 3 and 4 below) to compute the relative level of toxicity for a treatment condition.

a. The starting population is comprised of unexposed cells at the beginning of the recovery period

b. Exposed microcolonies collected at the end of the recovery period constitute the final population for the treatment. A population of unexposed cells is also collected at the same time as the final population for the control condition

3. An estimate of excess growth for a final population is performed as follows:
a. Estimate the portion of microcolonies that have grown in excess of the starting population by subtracting the microcolony size distribution of the final population by that of the starting population Specifically, for each microcolony size, the relative microcolony frequency of the final population is subtracted by the relative microcolony frequency of the starting population. If the subtraction result is positive, we define this value to be the relative frequency of microcolonies that have grown in excess of the starting population (excess microcolonies).
b. The relative frequencies of the excess microcolonies are multiplied by their corresponding microcolony size, and all the results are summed up to yield an estimate of the relative number of cells in these excess microcolonies. We define this estimate to be the excess growth of a treatment condition.

4. The excess growth of a treatment condition is divided by the excess growth of an untreated control condition (unexposed cells collected in parallel with exposed cells) to yield the relative toxicity.

Any of the foregoing sets of instructions (or steps) may be provided as a program which directs a computer (or computer system) to execute such steps. The program may be provided on computer-readable medium. Similarly the input data and output data may also be provided on computer-readable medium, and/or the latter may be displayed on a display device, e.g., a liquid crystal display panel or organic electroluminescence (EL) display panel.

The computer system may comprise a memory component. Such memory component may comprise a non-volatile storage medium, such as a hard disk or flash memory, and a volatile storage medium, such as dynamic random access memory (DRAM) or static random access memory (SRAM), and the like. This memory component may store data relating to the microwells and/or microcolonies, whether individually or collectively, and as well as image data including microscopic image data captured by the imaging device (e.g., the epifluorescence microscope). These image data, may include fluorescent images captured after fluorescent-staining the microcolonies. Notification parameters relating to the agent to which the microwells were exposed, as well as the location of particular microwells (or pluralities of microwells) may also be stored in the memory component. Determination parameters for determining for example the amount of fluorescent signal from a microcolony may also be stored in the memory component. Furthermore, programs that are executed by the control part of a computer system are also stored in the memory component. Various computational results performed by the control part may also be temporarily stored in the memory part. The control part may be for example a processor that executes the various computational processing of the control device. A portion or all of these functional parts that constitute the control part may be functional hardware parts, such as large scale integration (LSI) or application specific integrated circuit (ASIC).

The “computer system” described here may include an OS or hardware such as peripheral equipment. In addition, “computer systems” are assumed to include home page providing environments (or display environments) when a WWW system is used.

A “computer-readable recording medium” may refer to a writable nonvolatile memory such as a flexible disk, a magneto-optical disk, a ROM, or a flash memory, a portable medium such as a CD-ROM, or a storage part such as a hard disk built into the computer system. Further, “computer-readable recording mediums” also include mediums which hold programs for a certain amount of time such as the volatile memory (for example, DRAM) and non-volatile memory inside a computer system serving as a server or a client when a program is transmitted via a network such as the internet or a communication line such as a telephone line. Computer-readable recording mediums include non-transitory media.

In addition, the programs described above may be transmitted from a computer system in which the program is stored in a storage part or the like to another computer system via a transmission medium or by means of transmission waves in a transmission medium. Here, a “transmission medium” for transmitting a program refers to a medium having a function of transmitting information, as in the case of a network (communication network) such as the internet or a communication line (communication wire) such as a telephone line. In addition the program described above may be a program for realizing some of the functions described above. Further, the program may be a so-called differential file (differential program) capable of realizing the functions described above in combination with programs already recorded in the computer system.

### Commercial Applications

General utility-toxicity testing for growth inhibition. ToxChip can be used in general as a toxicity assay, which is often used for screening of biologically harmful chemicals. Testing for cell sensitivity is very commonly done in drug discovery to predict side effects of a drug treatment (through providing a predictive evidence of compound safety). Biological impact of environmental agents is a growing area of research that can benefit from high-throughput methods like ToxChip for toxicity assessment. ToxChip provides a simple, sensitive, rapid, and inexpensive means to determine whether a compound can affect the ability of cells to grow and to form colony. ToxChip's performance is comparable to the gold-standard method for toxicity testing, the cologenic assay, which is laborious and time consuming.

High throughput toxicity screening for pharmacological agents, environmental particles, or bioactive compounds. Toxicity of test compounds can be measured by quantifying their ability to inhibit cell growth. Multiple treatment conditions, such as different dosages of drug, treatment time, and timing for drug exposure, can be performed concurrently on the same chip because treatment conditions can be easily separated by a 96-well bottomless plate. Similarly, the study of multiple cell lines at the same time is also possible. Since assay turnover time is only one week, many tests can be conducted in a short period of time.

In vitro assessment of cancer treatment. The efficacy of a chemotherapeutics or radiation to kill cancer cells or to inhibit cancer cell proliferation can be quickly assessed. This is important to predict the treatment outcome and treatment efficacy, which can be improved by using the right dose. Side effects of chemotherapy can be reduced as well.

Amenable to common high-throughput screening systems. ToxChip can be coupled with live-cell imaging systems (e.g., epifluorescence microscopes equipped with cell culture chambers) to study real-time cell growth kinetics and production of survival factors or apoptotic factors tagged with fluorescence proteins. The multi-well platform of ToxChip is compatible with liquid handling robots and Cellomics systems for automated fluorescence imaging and quantitative analysis. This enables large-scale screens for toxicity of compound libraries.

Simultaneous screening of growth inhibition and genotoxicity. ToxChip and CometChip assays can be combined to study cell proliferation/cell death and DNA damage on the same platform since both these assays use the same platform of agarose chips. CometChip measures the level of DNA damage in the cells by quantifying the level of DNA in comet tail after electrophoresis while ToxChip measures the survival of the cells by quantifying the total DNA intensity. Total DNA intensity still can be measured even after electrophoresis. Combining these two assays gives information on both growth inhibition and genotoxicity of a toxic agent.

Use ToxChip to quantify cell division ratio. Cells transfected with histone H2B gene fused with green fluorescent protein (GFP) (H2B-GFP) can be used to study cell cycle effect of compounds. The principle behind it is to analyze the H2B-GFP expression that reduced with each division. By analyzing GFP expression, a population of cells exhibiting a 2-fold reduction in GFP fluorescence, reflective cell division, is detected. However, if a compound affects cell division, more H2B-GFP retention will be observed in cells. Combining this technique with Toxchip allows the kinetics of the toxicity to be studied. For example, if compound-treated cells do not form colony (low DNA intensity), but expressed high signal of GFP (from H2B), it is known that the compound interferes cell division and not through cell death pathway.

Another method that is commonly used to study cell cycle effect is by using thymidine analog EdU that is able to incorporated into newly synthesized DNA in proliferating cells. Signal increases with new DNA synthesis and is usually detected using fluorescence or absorbance. Similarly, this assay can be coupled with ToxChip to study relationship between toxicity and cell division.

Use ToxChip to study cell cycle effects of a compound/exposure. The traditional method used for cell cycle analysis is to stain cellular DNA quantitatively with DNA-binding dye such as propidium iodine (PI). The florescence intensity of the stained cells correlate with the amount of DNA they contain. When cells are in S phase, DNA content duplicates and have more DNA than cells in G1, until they doubled their DNA content and cells in G2 will be twice as bright as cells in G1. Flow cytometry enables single cell analysis and reveals distribution of cells in the three major phases of the cycle (G1, S and G2/M), and make it possible to detect apoptotic cells that contain fragmented DNA. However, this single time-point measurement only reveals percentage of cells in different phase and not providing information on cell cycle kinetics.

ToxChip can be used as a platform for cell cycle effect analysis. Cells on ToxChip are dividing (forming colony) while being labeled with DNA-binding dye. Staining cells directly on the chip minimize the stress placed upon on cells, as compared to preparing cells for flow cytometry assay (e.g., trypsinizing adherent cells, vortexing, transferring from tube to tube). Handling of cells may cause cell cycle arrest, and confound the results.

ToxChip analysis is impacted by cell cycle. Cells with a high percentage of S phase cells will have more DNA/cell than a culture with all cells in G1. This limitation is in common with all the other toxicity assays, except for the clonogenic assay.

High-throughput assessment of cell transformation. Anchorage-independent growth is one of the hallmarks for neoplastic transformation. ToxChip is inherently based on agarose and therefore can he used to screen for occurrence of anchorage-independent proliferation in cells that normally require attachment to external ligands.

Population studies. Because of its high-throughput capacity and its multi-log sensitivity, we anticipate that the ToxChip platform can be applied to detect subtle differences between people in population studies. Information about inter-individual variability in sensitivity to toxic exposures is useful in developing personalized disease treatment as well as in understanding risk factors in our environment. Studies have shown a wide range of variation in sensitivity toward different DNA damaging agents in lymphoblastoid cell lines derived from genetically diverse healthy individuals (5, 27) Many studies have also observed differences in radiosensitivity among mitogen-stimulated T lymphocytes obtained from different individuals (28-30). We anticipate that ToxChip is well suited to study limited dividing cells, such as mitogen-stimulated lymphocytes., because of its ability to yield sensitive measurements after only a few days in culture.

The following Examples are included for purposes of illustration and are not intended to limit the scope of the invention.

### EXAMPLES

### Example 1

### Application of ToxChip for Analysis of BCNU-Induced Toxicity

To compare ToxChip to standard toxicity assays, TK6 cells were treated with a DNA damaging agent N,N′-bis(2-chloroethyl)-N-nitrosourea (BCNU). BCNU is a chemotherapeutic agent that induces extremely cytotoxic DNA lesions, including inter-strand crosslinks (14).

(DNA crosslinks are formed via a series of chemical reaction steps, which start with the generation of O⁶-chloroethylguanine lesions. O⁶-methylguanine methyl transferase (MGMT) protein removes the chloroethyl group from the O⁶ position of guanine (16, 17). The lymphoblastoid TK6 cells are deficient in MGMT and thus are very sensitive to BCNU treatment (5).

TK6 cells were loaded into the micro wells of the ToxChip. After 48 hours in media at 37C with 5% CO₂, cells were exposed to BCNU solubilized in serum-free media at the indicated concentrations. After one hour, the ToxChip was rinsed with PBS and media was replaced. Cells were then allowed to grow under tissue culture conditions for up to 72 hours. The total culture time from initial loading to the final analysis can be 120 hours or longer.

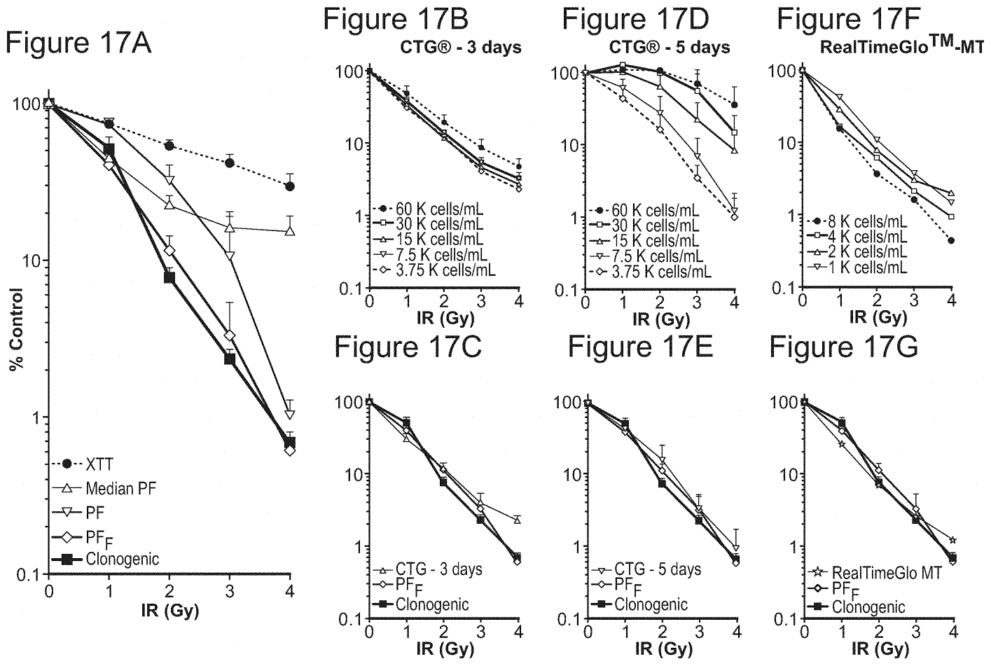

For colony size analysis using ToxChip, the distribution of F/M was quantified. FIG. 16 shows that the BCNU treated cells are less able to form larger colonies, as reflected by lower FM.

### Example 2

### γ-Ray Survival Curves: Comparison of ToxChip, XTT, CellTiter-Glo® (CTG®) and Liquid Colony Formation Assay

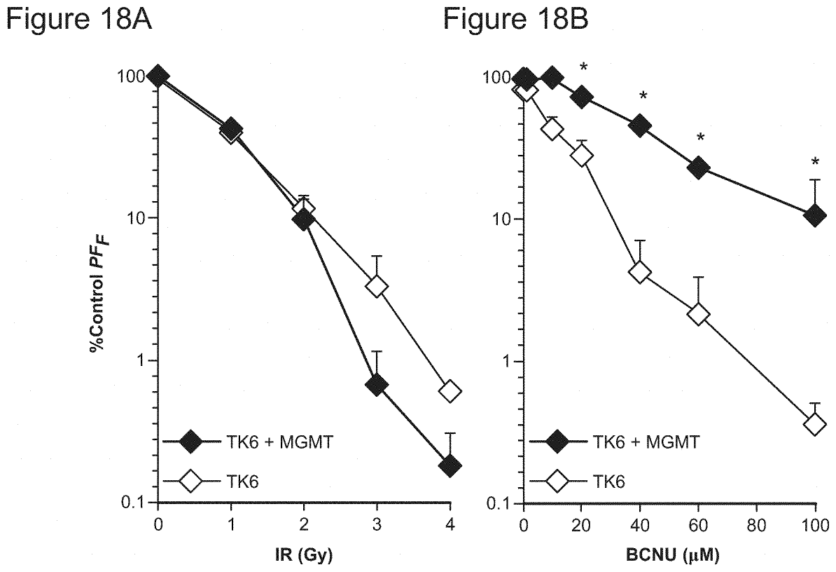

Having defined PF and PF_{F} as different parameters to quantify toxicity using ToxChip, we wanted to learn about the sensitivity of each parameter in measuring toxicity. A parallel analysis using ToxChip and two standard growth assays was performed to measure the sensitivity of TK6 cells to γ-radiation. We compared the ToxChip approaches using median F/M, PF, and PFF to the XTT assay, the CTG® assay, the RealTime-Glo™ MT assay, and the liquid colony formation assay (7). The XTT method is a widely used colorimetric assay that estimates the number of viable cells by measuring the cell's ability to reduce the faint yellow salt (2,3-bis-(2-methoxy-4-nitro-5-sulfophenyl)-2H-tetrazolium-5-carboxanilide) (XTT) to a bright orange formazan dye (8). Here we show that the XTT assay captured the exponential reduction in TK6 cell viability with increasing doses of γ-radiation (FIG. 17A). However, compared to the results in previously published studies (1-4), the XTT assay appears to be ˜2 orders of magnitude less sensitive. The liquid colony forming assay shows a steep decrease in viability over more than two orders of magnitude when moving from 0 to 4 Gy (FIG. 17), results that are consistent with the literature (1-4). It is interesting that despite the loss in sensitivity, the XTT assay is used far more frequently than the colony forming assay due to the large volumes of media and laboriousness of data collection.

Using the median F/M parameter for ToxChip, we observed significantly more toxicity than the results from the XTT assay (FIG. 17). However, the median F/M significantly underestimates TK6 's sensitivity to γ-radiation compared to the liquid colony formation assay (p<0.05 for 2-4 Gy, student t-test, 2-tailed, unequal variance). In contrast to the median F/M, we observed that the PF values reveal significantly more toxicity, though it is not as sensitive as the colony forming assay. Remarkably, the PF_{F} parameter yielded comparable toxicity results with the liquid colony forming assay. We did not detect any significant difference between the results from the PF_{F} analysis and the liquid colony forming assay (p>0.05 for all doses, student t-test, 2-tailed, unequal variance). Importantly, the results from the PF_{F} analysis are also consistent with previously published studies (1-4).

The CTG® assay (from Promega) is based on luminescent quantification of cellular ATP as a measure of metabolically active cells (9). Specifically, beetle luciferin is added to lysed cells. ATP is rate limiting for beetle luciferin to be enzymatically converted to oxyluciferin by firefly luciferase, with the output of light. Thus, the amount of ATP in a sample (proportional to number of metabolically active cells) can be estimated by the extent to which light is emitted. In order to test CTG®'s robustness against cell plating densities, γ-irradiated TK6 cells were plated at different cell densities, three of which were not expected to reach confluency in 5 days. CTG® analyses were performed after 3 or 5 days in culture. While plating density has no effects on day 3 (FIG. 17B), by day 5, γ ray-induced toxicity appears to decrease with higher densities (FIG. 17D). The dependence on cell plating density poses an important limitation for CTG® compared to ToxChip for the following two reasons. First, for ToxChip, the number of cells per microwells follows a Poisson distribution with an average number of 3 cells per well and a maximum of 6 cells per well. Therefore the “plating density” for ToxChip stays relatively consistent regardless of the cell density in the loading suspension. Second, because ToxChip uses a very small number of cells per chip (maximum 80,000 cells for a chip the size of a 96 well plate), we believe the changes in culture media due to cell growth (e.g., nutrient depletion and accumulation of metabolic waste) are negligible for at least four days of culture and do not impair cell growth rate (FIG. 9B, ToxChip line).

Looking at the CTG® results from the lowest plating density (3.75 K cells/mL), the CTG® assay significantly underestimates toxicity at 4 Gy compared to the clonogenic assay and the PF_{F} approach when the cells were analyzed 3 days post irradiation (p<0.05 for 4 Gy, student t-test, 2-tailed, unequal variance) (FIG. 17C). The underestimation of toxicity by CTG® has been previously documented. When the incubation time post irradiation was extended to 5 days, the result becomes more consistent with the clonogenic assay and the PF_{F} approach but only for the lowest cell plating density (FIG. 17E), further emphasizing the role of cell plating density in CTG®. It is also worth noting that although the CTG® assay is equally sensitive to the clonogenic and ToxChip assays (FIG. 17E), it requires the cells to be lysed in order to liberate the cytosolic ATP. Hence, the cells cannot be used in combination with other assays, such as LIVE/DEAD staining or Annexin V staining.

The PF_{F} approach was compared to a recently developed assay from Promega, the RealTime-Glo™ MT assay. Like CTG®, RealTime-Glo™ MT measures luminescence as its output. Similar to MTT/XTT, RealTime-Glo™ MT estimates the number of metabolically active cells by measuring the cells' reducing potential. The assay uses the MT Cell Viability substrate that can be reduced by live cells to a NanoLuc® substrate. The NanoLuct substrate diffuses outside the cells and is used by NanoLuc® Enzyme to produce light (Promega, RealTime-Glo™ MT Cell Viability Assay technical manual). The RealTime-Glo™ MT assay eliminates the need for cell lysis (CTG® assay) and is compatible with parallel or downstream analyses by other assays. Promega also claims that the assay's reagents are not cytotoxic for up to 3 days; therefore, cell growth can be monitored in real-time. The preliminary experiments outlined herein show that 3 days after γ-ray treatment, the results appear to be dependent on cell plating density (FIG. 17F). The Real Time-Glo™ MT data obtained from averaging all cell plating densities appear to show comparable sensitivity to the results from the clonogenic assay and the PF_{F} approach (FIG. 17G). However, because the RealTime-Glo™ MT assay also measure the cells' reducing potential like the MTT/XTT assays, we expect that there can be artifacts that come from changes in pH, factors that affect cellular metabolism, and constituents in cell media (e.g. reducing agents) (8, 10, 11).

We conducted further experiments to investigate the sensitivity of μCC in measuring toxicity, we measured the toxicity of γIR to TK6 cells using μCC and other existing methods in parallel. Specifically, we compared μCC to the colony formation assay (7) and to two commercially available methods, XTT and CTG®. γ-irradiated TK6 microcolonies on μCC were analyzed 3-4 days after exposure. The recovery periods for the other methods were varied to maximize their sensitivity.

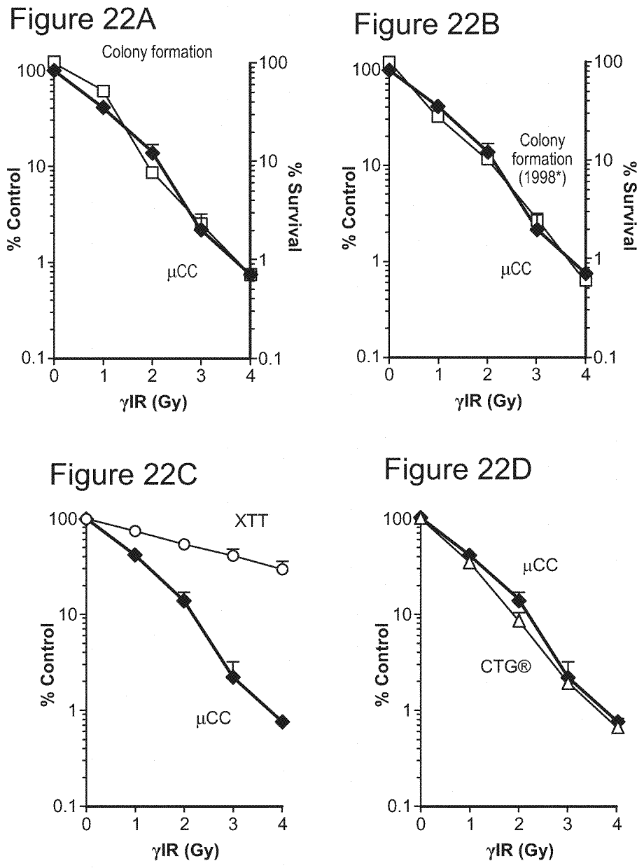

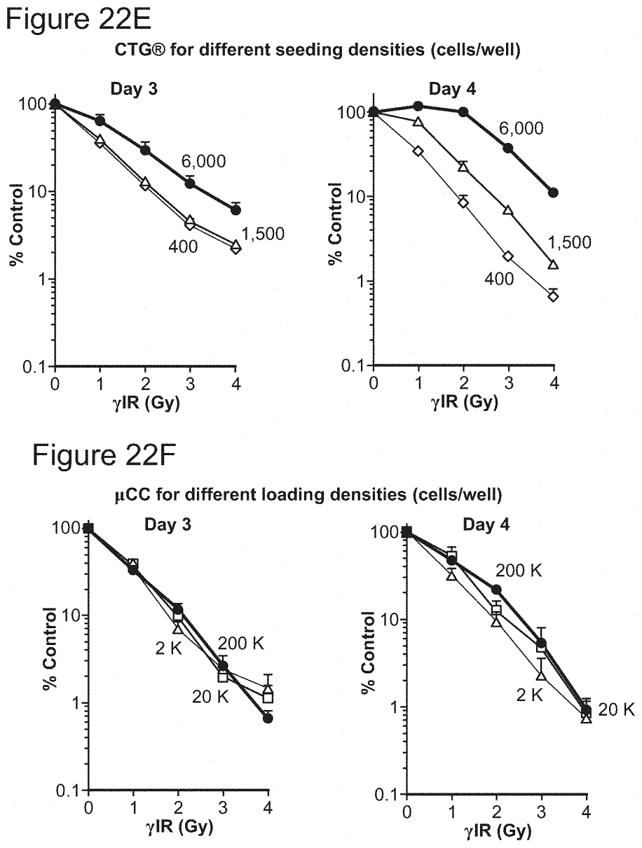

We performed a direct comparison between the μCC approach and the gold standard colony formation assay (7). For the colony formation assay, γ-irradiated TK6 cells were analyzed for colony formation in microtiter plates 3 weeks after exposure. We tracked the appearance of colonies in 96-well plates over 18 days and observed that some colonies were not obvious until the last day (FIG. 21). Therefore, we decided on a timescale of 3 weeks before counting the colonies in order to maximize the assay's sensitivity. In contrast, μCC data were obtained 3-4 days after y irradiation. Remarkably, μCC yields an exponential toxicity curve undistinguishable from the survival curve obtained from the colony formation assay (FIG. 22A). The result from μCC is also consistent with previously published studies using the colony formation assay (1-4), FIG. 22B). We concluded that it is possible for μCC within a few days to measure toxicity with a high level of sensitivity and multi-log dynamic range similar to the gold standard colony formation assay.

Because of the laborious, time- and resource-consuming nature of the colony formation assay, many microtiter-based high-throughput methods for measuring toxicity have been developed. We sought to compare μCC was compared with two of the most popular commercially available assays, XTT and CTG®. The XTT method is a widely used colorimetric assay that estimates the number of viable cells by measuring the cell's ability to reduce the faint yellow salt (2,3-bis-(2-methoxy-4-nitro-5-sulfophenyl)-2H-tetrazolium-5-carboxanilide) (XTT) to a bright orange water-soluble formazan dye (8). When the γ-irradiated TK6 cells on microtiter plates were analyzed with XTT three days after exposure, an exponential reduction in TK6 cell growth was observed (FIG. 22C). Notably, μCC displays a dynamic range 2 orders of magnitude more than XTT.

The CTG® assay (from Promega) is based on luminescent quantification of cellular ATP as a measure of metabolically active cells (9). Specifically, beetle luciferin is added to lysed cells. ATP is rate limiting for beetle luciferin to be enzymatically converted to oxyluciferin by firefly luciferase, with the output of light. Thus, the amount of ATP in a sample (proportional to number of metabolically active cells) can be estimated by the extent to which light is emitted. Because an extended recovery period after a toxic exposure (5 to 7 days) has been generally recommended to maximize the sensitivity of CTG® (109), we anticipated that cell plating density might affect the final measurements of toxicity due to changes in cellular metabolism in long-term cultures. In order to test the robustness of CTG® against cell plating densities, we seeded γ-irradiated. TK6 cells at different cell densities in microtiter plates. We performed CTG® analyses after 3 or 4 days in culture. While the lowest plating density (˜400 cells/well) analyzed after 4 days with CTG® yields toxicity levels highly similar to those measured by μCC (FIG. 22D), toxicity measured by CTG® appears to decrease with higher cell seeding densities (FIG. 22E). This effect of cell seeding densities is especially pronounced for the longer recovery period (FIG. 22E, right).

To evaluate the robustness of μCC compared to CTG®, we also measured γIR toxicity using three different cell loading densities per macrowell and two different recovery periods of 3 and 4 days. Because the microwells are fixed in size, the number of TK6 cells loaded into each microwell is restricted within a range between 1 and 7 cells regardless of the cell loading density per macrowell. Therefore, we anticipated that cell loading densities would have a relatively small effect on μCC results. As expected, in contrast to the results from CTG® (FIG. 22F), γIR-induced toxicity in TK6 cells measured by μCC is robust against a wide range of cell loading density (spanning 2 orders of magnitude) and is consistent for both 3-day and 4-day recovery periods (FIG. 22F).

### Example 3

### The Role of DNA Repair in Cell Survival

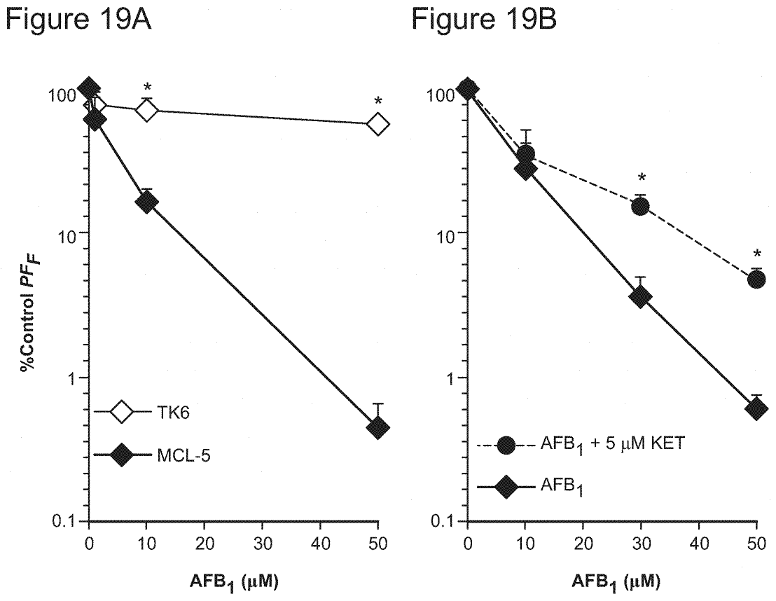

Viability assays are often used in study DNA repair genes and their roles in toxicity induced by different types of DNA damage. As an example of how ToxChip can be used for study of the role of DNA repair, we applied the PF_{F} method to measure differential sensitivity of TK6 and TK6+MGMT cells to N,N′-bis (2-chloroethyl)-N-nitrosourea (BCNU) and γ-radiation. BCNU is an alkylating agent that is a chemotherapeutic used to treat brain cancers (5, 12, 13). BCNU induces highly cytotoxic DNA inter-strand crosslinks (14). DNA crosslinks are formed via a series of chemical reaction steps, which start with the generation of O6-chloroethylguanine lesions, which then react a second time with bases on the opposite strand (15). It is known that the O6-methylguanine methyl transferase (MGMT) protein prevents the formation of highly toxic interstrand crosslinks (16, 17). The lymphoblastoid TK6 cells are deficient in MGMT and have been shown to be very sensitive to BCNU toxicity (5). The TK6+MGMT cells are TK6 cells stably transfected with cDNA expressing the protein MGMT and have been reported to be significantly more resistant to BCNU than the TK6 cells (5, 16, 17). As a control, we studied the effects of γ-radiation, for which MGMT is not expected to play a role. Results show that there is no significant difference between TK6 and TK6+MGMT (FIG. 18A), which is consistent with the fact that MGMT does not repair strand breaks induced by γ-radiation. In contrast, there is a significant difference of approximately 1 log between TK6 and TK6+MGMT when challenged with BCNU, which is consistent with previous literature (FIG. 18B). Taken together, these results show that ToxChip yields similar results to the published literature and emphasize that ToxChip can be used to assess DNA repair genes on DNA damage-induced cytotoxicity.

Endogenously, DNA is constantly under risk of damage from reactive products of cellular metabolism and inflammatory response. DNA is also continuously exposed to damaging agents from exogenous sources (e.g., UV-radiation, smoke, byproducts from food processing, reactive metal species). DNA damage can disrupt DNA replication and transcription, which can ultimately lead to cell death and mutations that promote cancer and premature aging. To combat problems posed by DNA damage, cells have evolved a network of DNA repair responses. Understanding the molecular mechanism of DNA repair pathways is essential in assessing human genetic risk factors in response to environmental exposures.

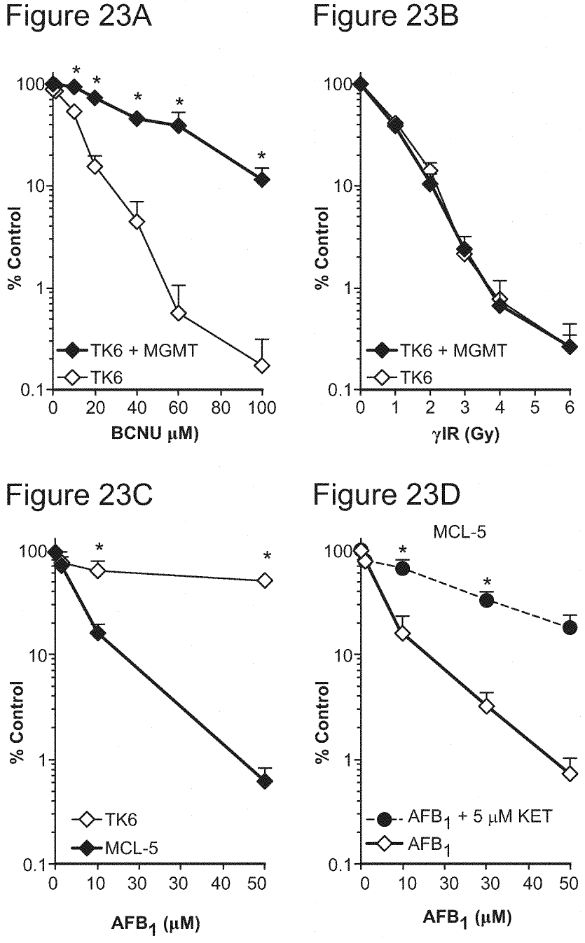

As an example of how μCC can be used for studies of DNA repair, we applied wEF to measure differential sensitivity of cells to N,N′-bis (2-chloroethyl)-N-nitrosourea (BCNU) and γ-radiation. BCNU is an alkylating agent that is a chemotherapeutic used to treat brain cancers (5, 12-13). BCNU induces highly cytotoxic DNA inter-strand crosslinks (14). DNA crosslinks are formed via a series of chemical reaction steps that start with the generation of O⁶-chloroethylguanine lesions, which then react a second time with bases on the opposite strand (15). It is known that the O⁶-methylguanine methyl transferase (MGMT) protein prevents the formation of highly toxic interstrand crosslinks (16, 17). The lymphoblastoid TK6 cells are deficient in MGMT and have been shown to be very sensitive to BCNU toxicity (5). The TK6+MGMT cells are TK6 cells stably transfected with cDNA expressing the MGMT protein and have been reported to be significantly more resistant to BCNU than the TK6 cells (5, 16, 17). As expected, there is a significant difference in sensitivity to BCNU between TK6 and TK6+MGMT, which is consistent with previous literature (5) (FIG. 23A). As a control, we studied the effects of γ-radiation, for which MGMT is not expected to play a role, were studied. Results show that TK6 and TK6+MGMT are similarly sensitive to γIR-induced toxicity (FIG. 23B), consistent with the fact that MGMT is not involved in strand break repair induced by γ-radiation.

### Example 4

### Analysis of Toxicity of Xenobiotics in Metabolically Relevant Conditions

Viability assays are widely used to monitor potential health impact of industrial and pharmaceutical chemicals. A major drawback of current in vitro viability assays is the lack of an appropriate cell model that can provide xenobiotic metabolic capacity. Xenobiotics are extensively metabolized in the human body. This process can result in reactive intermediates that can form adducts with DNA and proteins, which may lead to mutations, tumorigenesis, and cell death (18). It is, therefore, essential to assess the toxicity of chemicals in metabolically relevant conditions. The cytochromes P450 (CYP450s) are a superfamily of metabolizing enzymes, responsible for 70-80% of phase I metabolism in the liver (19). To enhance the utility of ToxChip in measuring toxicity of both parent chemicals and their metabolites, we incorporated the use of MCL-5, a metabolically competent cell line. MCL-5 is a human B-lymphoblastoid cell line that has been engineered to stably express human cytochrome P450 CYP1A1 CYP1A2, CYP2A6, CYP2E1, CYP3A4, and microsomal epoxide hydrolase (mEH) (20). Together, these metabolic enzymes arc responsible for the metabolism of ˜50% phase I metabolism of many common xenobiotics (18, 19).

Millions of people worldwide are exposed to aflatoxin B₁ (AFB₁), which following metabolic activation creates multicyclic DNA adducts that induce DNA damage that promotes cancer (21). AFB₁ is present in a mold present on grain. In combination with infection with hepatitis B, AFB₁ causes and approximately 60-fold increase in the risk of liver cancer, and is thus the major cause of cancer in many regions of the world (22). The metabolite AFB₁ evo-8,9-epoxide, generated by oxidation of AFBI mainly by CYP3A4, is reactive with DNA and has been shown to be mutagenic and carcinogenic (23-25).

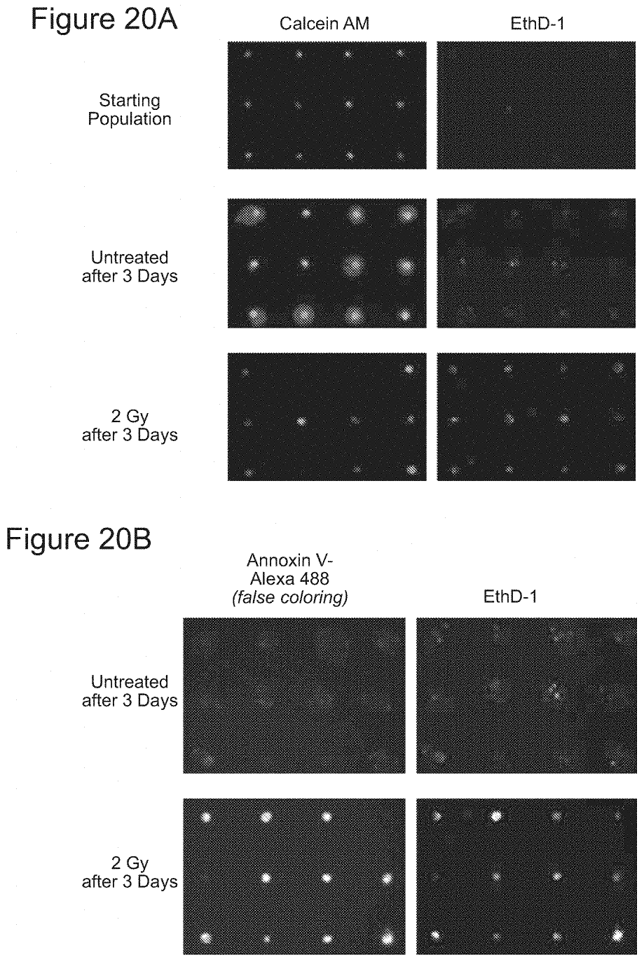

To test ToxChip's application in measuring metabolism-induced toxicity, we treated MCL-5 cells with AFB₁ on ToxChip and analyzed their PF_{F}s three days post treatment. MCL-5 cells stably express CYP3A4 and have been shown to be highly sensitive to AFB₁ (20). To control for the effects of AFB₁ metabolism, we included TK6 as a negative control cell line. We expected TK6 cells to be relatively insensitive to AFB₁ treatment due to their low enzymatic capacity to metabolize the procarcinogen. As an additional control, we co-treated MCL-5 cells with ketoconazole (KET), a well-known potent inhibitor of CYP3A4 activity (26). FIG. 19A shows TK6 cells are relatively unaffected by AFB₁ treatment and are significantly less sensitive compared to MCL-5 cells. When CYP3A4 activity in MCL-5 cells is inhibited by KET, we saw significant rescue of MCL-5 cells following treatment of AFB₁, further supporting that CYP3A4 activity in MCL-5 cells drives the observed AFB₁ toxicity is (FIG. 19B). Taken together, the incorporation of MCL-5 cells in ToxChip yields a rapid and sensitive method to test for toxicity of xenobiotics in a metabolically relevant context.

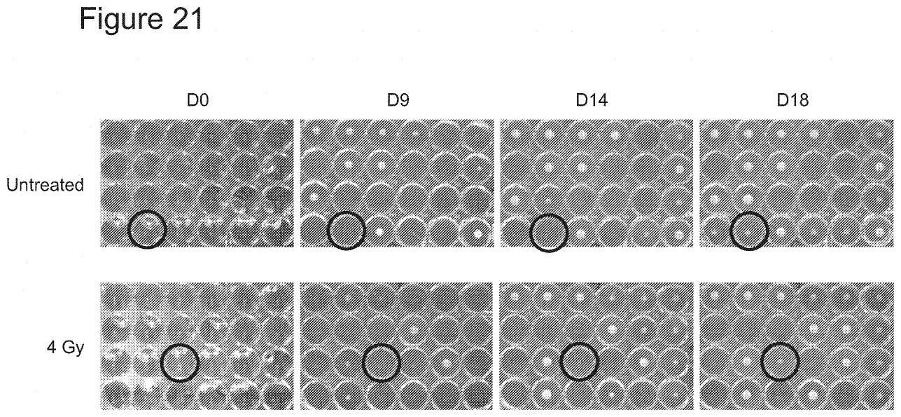

While the results here demonstrate the sensitivity and efficacy of the ToxChip for evaluating the effects of genotoxic exposures, the current assay does not show the proportion of dead cells that are in a colony. Importantly, this is also true for the colony forming assay. Nevertheless, we anticipated that being able to discern live versus dead cells within colonies could provide the knowledge about the extent to which cells are dead and could be useful in revealing the underlying cause of cell death. To gain additional insights, we explored the utility of a live-dead stain for discerning the extent to which colonies contain dead cells. To distinguish between live and dead cells, 3 days after γ-radiation, TK6 microcolonies cultured on ToxChip were stained with calcein-AM and ethidium homodimer-1. As expected, the untreated population had a very low level of cell death, as shown in FIG. 20A (middle panels) where only a few cells in several colonies stained red. In contrast, the 2 G y-irradiated population exhibited a high level of cell death across colonies (FIG. 20A, bottom panels), demonstrating that the small colony sizes are due to cytotoxic effects. In addition to live-dead analysis, it is also possible to analyze the Annexin V signal to detect apoptosis (FIG. 20B). Taken together, it has been demonstrated herein that viability staining methods can be incorporated in ToxChip, potentially enabling the ability to distinguish between cytotoxic and cytostatic effects (e.g. irreversible cell cycle arrest) and extending the applicability of ToxChip to non-dividing cells.

Viability assays are widely used to monitor potential health impact of industrial and pharmaceutical chemicals. A major drawback of current in vitro viability assays is the lack of an appropriate cell model that can provide capacity for metabolisms of foreign substances (xenobiotics). In the human body, xenobiotics are extensively metabolized, mainly by hepatocytes in the liver. This process can result in reactive intermediates that can form adducts with DNA that may lead to mutations, tumorigenesis, and cell death (18). It is, therefore, essential to assess the toxicity of chemicals in metabolically relevant conditions. The cytochromes P450 (CYP450s) are a superfamily of metabolizing enzymes, responsible for 70-80% of phase I metabolism in the liver (19). To provide μCC with the ability to measure toxicity of both parent chemicals and their metabolites, we incorporated a metabolically competent cell line, MCL-5. MCL-5 is a human B-lymphoblastoid cell line that has been engineered to stably express human cytochrome P450 CYP1A1 CYP1A2, CYP2A6, CYP2E1, CYP3A4, and microsomal epoxide hydrolase (mEH) (20) Together, these metabolic enzymes are responsible for approximately 50% of P450 activity in phase I metabolism (19).

Millions of people worldwide are exposed to aflatoxin B₁(AFB₁), a procarcinogen present in certain molds (Aspergillus flavus and Aspergillus parositicus) usually found in grains, in combination with hepatitis B infection, AFB₁ has been reported to increase the risk of liver cancer by approximately 60-fold, and is thus the major cause of cancer in many regions of the world (22). Studies have shown AFB₁ is metabolized by a number of P450 enzymes. The most genotoxic metabolite, AFB₁ exo-8,9-epoxide, is generated via oxidation of AFB mainly by CYP3A4 (35-37). AFB₁ exo-8,9-epoxide is highly unstable and readily reacts with guanine to form a number of bulky DNA adducts that can lead to mutations and carcinogenesis (23-25).

To test μCC's application in measuring metabolism-induced toxicity, we treated MCL-5 cells with AFB₁ and analyzed for their EG values three days post treatment. MCL-5 cells stably express CYP3A4 and have been shown to be highly sensitive to AFB₁ (20). To control for the effects of AFB₁ metabolism, we included TK6 as a negative control cell line. We expected TK6 cells to be relatively insensitive to AFB₁ treatment due to their low enzymatic capacity to metabolize the procarcinogen. As an additional control, we co-treated MCL-5 cells with ketoconazole (KET), a well-known potent inhibitor of CYP3A4 activity (26). FIG. 23C shows TK6 cells are relatively unaffected by AFB₁ treatment and are significantly less sensitive compared to MCL-5 cells (more than 2-log difference at the highest AFB₁ dose). When CYP3A4 activity in MCL-5 cells is inhibited by KET, we saw a significant rescue of MCL-5 cells following treatment of AFB₁, further supporting that AFB₁ metabolism by CYP3A4 drives the observed toxicity(FIG. 23D). Taken together, the incorporation of MCL-5 cells in μCC yields a rapid and sensitive method to test for toxicity of xenobiotics in a metabolically relevant context.

### REFERENCES

1 Wenz F, Azzam E I, Little J B. The response of proliferating cell nuclear antigen to ionizing radiation in human lymphoblastoid cell lines is dependent on p53. Radiat Res. 1998;149(1):32-40. PubMed PMID: 9421152.
2. Schwartz J L, Jordan R, Sedita B A, Swenningson M J, Banath J P, Olive P L. Different sensitivity to cell killing and chromosome mutation induction by gamma rays in two human lymphoblastoid cell lines derived from a single donor: possible role of apoptosis. Mutagenesis. 1995; 10(3):227-33. PubMed PMID: 7666775.
3. Amundson S A, Xia F, Wolfson K, Liber H L. Different cytotoxic and mutagenic responses induced by X-rays in two human lymphoblastoid cell lines derived from a single donor. Mutat Res. 1993; 286(2):233-41. PubMed PMID: 7681535.
4. Schafer J, Bachtler J, Engling A, Little J B, Weber K J, Wenz F. Suppression of apoptosis and clonogenic survival in irradiated human lymphoblasts with different TP53 status. Radiat Res. 2002; 158(6):699-706. PubMed PMID: 12452772,
5. Valiathan C, McFaline J L, Samson L D. A rapid survival assay to measure drug-induced cytotoxicity and cell cycle effects. DNA Repair (Amst). 2012; 11(1):92-8. Epub 2011/12/03. doi: 10.1016/j.dnarep.2011.11.002. PubMed PMID: 22133811; PMCID: 3408220.
6. Hickman M J, Samson L D. Role of DNA mismatch repair and p53 in signaling induction of apoptosis by alkylating agents. Proc Natl Acad Sci USA. 1999; 96(19):10764-9.
7. Furth E E, Thilly W G, Penman B W, Liber H L, Rand W M. Quantitative assay for mutation in diploid human lymphoblasts using microliter plates. Anal Biochem. 1981; 110(1):1-8. PubMed PMID: 7011090.
8. Scudiero D A, Shoemaker R H, Paull K D, Monks A, Tierney S, Nofziger T H, Currens M J, Seniff D, Boyd M R. Evaluation of a soluble tetrazolium/formazan assay for cell growth and drug sensitivity in culture using human and other tumor cell lines. Cancer Res. 1988; 48(17):4827-33. PubMed PMID: 3409223.
9. Crouch S P, Kozlowski R, Slater K J, Fletcher J. The use of ATP bioluminescence as a measure of cell proliferation and cytotoxicity. J Immunol Methods. 1993; 160(1):81-8. PubMed PMID: 7680699.
10. Sumantran V N. Cellular chemosensitivity assays: an overview. Methods Mol Biol. 2011; 731:219-36. doi: 10.1007/978-1-61779-080-5_19. PubMed PMID: 21516411.
11. Herzog E, Casey A, Lyng F M, Chambers G, Byrne H J, Davoren M. A new approach to the toxicity testing of carbon-based nanomaterials—the clonogenic assay. Toxicol Lett. 2007; 174(1-3):49-60. doi: 10.1016/j.toxlet.2007.08.009, PubMed PMID: 17920791.
12. Hochberg F H, Parker L M, Takvori an T, Canellos G P, Zervas N T. High-dose BCNU with autologous bone marrow rescue for recurrent glioblastoma multiforme. J Neurosurg. 1981; 54(4).455-60. doi: 10.3171/jns.1981.54.4.0455. PubMed PMID: 6759300.
13. Durando X, Lemaire J J, Tortochaux J, Van-Praagh I, Kwiatkowski F, Vincent C, Bailly C, Verrelle P, Irthum B, Chazal J, Bay J O. High-dose BCNU followed by autologous hematopoietic stem cell transplantation in supratentorial high-grade malignant gliomas: a retrospective analysis of 114 patients. Bone Marrow Transplant. 2003; 31(7):559-64. doi: 10.1038/sj.bmt.1703889. PubMed PMID: 12692621.
14. Gonzaga P E, Potter P M, Niu T Q, Yu D, Ludlum D B, Rafferty J A, Margison G P, Brent T P. Identification of the cross-link between human O6-methylguanine-DNA methyltransferase and chloroethylnitrosourea-treated DNA. Cancer Research. 1992; 52(21):6052-8.
15. Tong W P, Kirk M C, Ludlum D B. Formation of the cross-link 1-[N3-deoxycytidyl),2-[N1-deoxyguariosinyl]ethane in DNA treated with N,N′-bis(2-chloroethyl)-N-nitrosourea. Cancer Research. 1982; 42(8):3102-5.
16. Kaina B, Fritz G, Mitra S, Coquerelle T. Transfection and expression of human O⁶-methylguanine-DNA methyltransferase (MGMT) cDNA in Chinese hamster cells: the role of MGMT in protection against the genotoxic effects of alkylating agents. Carcinogenesis. 1991; 12(10).1857-67.
17. Gerson S L. MGMT: its role in cancer aetiology and cancer therapeutics. Nat Rev Cancer. 2004; 4(4):296-307. doi: 10.1038/nrc1319. PubMed PMID: 15057289.
18. Nebert D W, Dalton T P. The role of cytochrome P450 enzymes in endogenous signalling pathways and environmental carcinogenesis. Nat Rev Cancer. 2006; 6(12):947-60. doi: 10.1038/nrc2015. PubMed PMID: 17128211.
19. Evans W E, Relling M V. Pharmacogenomics: translating functional genomics into rational therapeutics. Science. 1999; 286(5439).487-91. PubMed PMID: 10521338.
20. Crespi C L, Gonzalez F J, Steimel D T, Turner T R, Gelboin H V, Penman B W, Langenbach R. A metabolically competent human cell line expressing five cDNAs encoding procarcinogen-activating enzymes: application to mutagenicity testing. Chem Res Toxicol. 1991; 4(5):566-72. PubMed PMID: 1793807.
21. Essigmann J M, Croy R G, Bennett R A, Wogan G N. Metabolic activation of aflatoxin B1: patterns of DNA adduct formation, removal, and excretion in relation to carcinogenesis. Drug metabolism reviews. 1982,13(4):581-602. doi: 10.3109/03602538209011088. PubMed PMID: 6813091.
22. Henry S H, Bosch F X, Bowers J C. Aflatoxin, hepatitis and worldwide liver cancer risks. Adv Exp Med Biol. 2002; 504:229-33. PubMed PMID: 11922091.
23. Johnson W W, Guerigerich F P, Reaction of aflatoxin B1 exo-8,9-epoxide with DNA: kinetic analysis of covalent binding and DNA-induced hydrolysis. Proc Natl Acad Sci USA. 1997; 94(12):6121-5. PubMed PMID: 9177180; PMCID: PMC21012.
24. Smela M E, Hamm M L, Henderson P T, Harris C M, Harris T M, Essigmann J M. The aflatoxin B(1) formamidopyrimidine adduct plays a major role in causing the types of mutations observed in human hepatocellular carcinoma. Proc Natl Acad Sci USA. 2002; 99(10):6655-60. doi: 10.1073/pnas.102167699. PubMed PMID: 12011430; PMCID: PMC124458.
25. Kensler T W, Qian G S, Chen J G, Groopman J D. Translational strategies for cancer prevention in liver. Nat Rev Cancer. 2003; 3(5):321-9. doi: 10.1038/nrc1076. PubMed PMID: 12724730.
26. Crespi C L, Miller V P, Penman B W. Microtiter plate assays for inhibition of human, drug-metabolizing cytochromes P450. Anal Biochem. 1997; 248(1):188-90. doi: 10.1006/abio.1997.2145. PubMed. PMID: 9177742.
27. Fry R C, Svensson J P, Valiathan C, Wang E, Hogan B J, Bhattacharya S, Bugni J M, Whittaker C A, Samson L D. Genomic predictors of interindividual differences in response to DNA damaging agents. Genes Dev. 2008; 22(19):2621-6. doi: 10.1101/gad.1688508. PubMed PMID: 18805990; PMCID: PMC2559901.
28. West C M, Elyan S A, Berry P, Cowan R, Scott D. A comparison of the radiosensitivity of lymphocytes from normal donors, cancer patients, individuals with ataxia-telangiectasia (A-T) and A-T heterozygotes. Int J Radiat Biol. 1995; 68(2):197-203. PubMed PMID: 7658145.
29. Jones L A, Scott D, Cowan R, Roberts S A. Abnormal radiosensitivity of lymphocytes from breast cancer patients with excessive normal tissue damage after radiotherapy: chromosome aberrations after low dose-rate irradiation. Int J Radiat Biol. 1995; 67(5):519-28. PubMed PMID: 7775827.
30. Cole J, Arlett C F, Green M H, Harcourt S A, Priestley A, Henderson L, Cole H, James S E, Richmond F. Comparative human cellular radiosensitivity: II. The survival following gamma-irradiation of unstimulated (G0) T-lymphocyles, T-lymphocyte lines, lymphoblastoid cell lines and fibroblasts from normal donors, from ataxia-telangiectasia patients and from ataxia-telangiectasia heterozygotes. Int J Radiat Biol. 1988; 54(6):929-43. PubMed PMID: 2903890.
31. Weingeist D M, Ge J, Wood D K, Mutamba J T, Huang Q, Rowland E A, Yaffe M B, Floyd S, Engelward B P. Single-cell microarray enables high-throughput evaluation of DNA double-strand breaks and DNA repair inhibitors. Cell cycle 2013; 12: 907-915.
32. Olive P L, Banath J P. The comet assay: a method to measure DNA damage in individual cells. Nature protocols 2006; 1: 23-29.
33. Wood D K, Weingeist D M, Bhatia S N, Engelward B P. Single cell trapping and DNA damage analysis using microwell arrays. Proceedings of the National Academy of Sciences of the United States of America 2010; 107: 10008-10013.
34. Franken N A, Rodermond H M, Stap J, Haveman J, van Bree C. Clonogenic assay of cells in vitro. Nature protocols 2006; 1: 2315-2319.
35. Guengerich F P, Johnson W W, Ueng T F, Yamazaki H, Shimada T. Involvement of cytochrome P450, glutathione S-transferase, and epoxide hydrolase in the metabolism of aflatoxin B1 and relevance to risk of human liver cancer. Environ Health Perspect. 1996; 104 Suppl 3:557-62. PubMed PMID: 8781383; PMCID: PMC1469621.
36. Gallagher E P, Kunze K L, Stapleton P L, Eaton D L. The kinetics of aflatoxin B1 oxidation by human cDNA-expressed and human liver microsomal cytochromes P450 1A2 and 3A4. Toxicology and applied pharmacology. 1996; 141(4595-606. doi: 10.1006/taap.1996.0326. PubMed PMID: 8975785.
37. Mace K, Aguilar F, Wang J S, Vautravers P, Gomez-Lechon M, Gonzalez F J, Groopman J, Harris C C, Pfeifer A M. Aflatoxin B1-induced DNA adduct formation and p53 mutations in CYP450-expressing human liver cell lines. Carcinogenesis. 1997; 18(7):1291-7. PubMed PMID: 9230270.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein, More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be to understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at, least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements, in general the term “or” as used herein shall only be interpreted, as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers. whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including.” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03

What is claimed is:

1. A method for monitoring cell growth in vitro comprising
loading cells in a plurality of microwells,
culturing the cells under conditions and for a time sufficient for cell growth and/or proliferation, thereby forming a microcolony in each microwell,
staining the microcolonies with a membrane-permeable DNA-specific fluorescent dye, and
imaging the microcolonies, thereby obtaining total fluorescent intensity per microcolony.

2. The method of claim 1, wherein the microwells are defined by a semi-solid matrix or solid matrix.

3. The method of claim 2, wherein the semi-solid matrix is agarose or other biologically compatible polymer.

4. The method of claim 3, wherein the agarose is normal melting point agarose.

5. The method of any one of the foregoing claims, wherein the plurality of microwells is provided in a fixed array of microwells.

6. The method of any one of the foregoing claims, wherein the plurality of microwells is physically partitioned from other pluralities of microwells.

7. The method of claim 6, wherein the plurality of microwells is physically partitioned by a macrowell of a bottomless 96 well plate.

8. The method of any one of the foregoing claims, wherein the number of cells initially loaded into the microwells is not uniform across the plurality and/or the number of cells initially loaded into microwells is not uniform between pluralities.

9. The method of any one of the foregoing claims, wherein cells in the microcolonies are not lysed before being stained.

10. The method of any one of the foregoing claims, wherein the cells are loaded into the microwells by gravity.

11. The method of any one of the foregoing claims, wherein the number of cells initially loaded into each microwell is in the range of 0-7 cells.

12. The method of any one of the foregoing claims, wherein the time sufficient for cell growth and/or proliferation is 1 day, 2 days, 3 days, or 4 days.

13. The method of any one of the foregoing claims, wherein the microcolonies are imaged using an epifluorescent microscope.

14. The method of claim 13, wherein a plurality of microcolonies are simultaneously imaged.

15. The method of claim 14, wherein 50-100 microcolonies are simultaneously imaged.

16. The method of any one of the foregoing claims, wherein the plurality of microwells are exposed to an agent after the cells are plated.

17. The method of claim 16, wherein the agent is a candidate growth-modifying agent or cytotoxic agent.

18. The method of any one of the foregoing claims, wherein the plurality of microwells is provided in a chip that comprises other pluralities of microwells, each plurality physically partitioned from other pluralities.

19. The method of claim 16, wherein a second plurality of microwells is not exposed to the agent.

20. The method of any one of the foregoing claims, wherein cells loaded into the microwells are layered with low melting point agarose.

21. The method of any one of the foregoing claims, wherein cells loaded into the microwells are layered with an extracellular matrix, which is then layered with low melting point agarose.

22. The method of any one of the foregoing claims, wherein the cells are adherent cells.

23. The method of any one of the foregoing claims, wherein the cells are non-adherent cells.

24. The method of any one of the foregoing claims, wherein the cells are a cell line.

25. The method of any one of the foregoing claims, wherein the cells are cancer cells.

26. The method of any one of the foregoing claims, wherein the cells are normal cells.

27. The method of any one of the foregoing claims, wherein the cells are human cells or bacterial cells.

28. The method of any one of the foregoing claims, wherein the total fluorescent intensity per microcolony comprises fluorescence intensity from live and dead cells in the microcolony.

29. The method of any one of the foregoing claims, wherein the microcolonies are non-clonal cell clusters each comprising 1-2000 cells.

30. The method of any one of the foregoing claims, wherein the DNA-specific fluorescent dye is a Vybrant DyeCycle dye, acridine orange, a SYTO nucleic acid stain, or a Hoechst stain.

31. A method for monitoring cytotoxic or growth inhibition effect of a compound on a population of cells comprising
loading cells in a plurality of semi-solid microwells,
exposing the cells to a candidate cytotoxic or growth inhibiting compound for a limited time,
culturing the cells under conditions and for a time sufficient for cell growth and/or proliferation, thereby forming microcolonies in each microwell,
staining the microcolonies with a membrane-permeable DNA-specific fluorescent dye,
imaging the microcolonies, thereby obtaining total fluorescent intensity per microcolony, and
measuring proliferation in the plurality of semi-solid microwells after exposure to the candidate cytotoxic or growth modifying (inhibiting or stimulating) compound.

32. The method of claim 31, wherein measuring proliferation comprises measuring proliferation fraction.

33. The method of claim 31, wherein measuring proliferation comprises measuring total proliferation fraction fluorescence.

34. The method of claim 31, wherein measuring proliferation comprises analysis of microcolony size distribution.

35. The method of any one of claims 31-34, wherein the microwells in a plurality comprise a non-uniform number of cells.

36. The method of any one of claims 31-35, wherein the microwells in a plurality each comprise 0-7 cells.

37. The method of any one of claims 31-36, wherein the microcolonies are non-clonal cell clusters each comprising 1-2000 cells.

38. The method of any one of claims 31-37, wherein the cytotoxic or growth inhibiting effect of a number of different compounds is monitored simultaneously using different pluralities of microwells provided in a single fixed array.

39. The method of any one of claims 31-38, wherein the microcolonies are stained without prior lysis of the cells.

40. A method for measuring proliferation in a cell population comprising
providing a fixed array of microwells arranged as physically partitioned pluralities of microwells,
loading cells into the microwells by gravity, wherein the number of cells between microwells of a plurality is not uniform,
exposing at least one plurality to a candidate cytotoxic or growth modifying compound, wherein at least one other plurality is not exposed to the candidate cytotoxic or growth modifying compound,
culturing the cells under conditions and for a time sufficient for cell growth and/or proliferation to form a microcolony per microwell,
measuring total DNA per microwell without lysing cells within the microwells, and
measuring proliferation fraction of treated cells relative to untreated cells.

41. The method of claim 40, further comprising measuring total proliferation fraction fluorescence of treated cells and untreated cells.

42. The method of claim 40 or 41, wherein the microwells are semi-solid microwells.

43. The method of any one of claims 40-42, wherein the total number of cells in each plurality is approximately equal between pluralities.

44. The method of any one of claims 40-43, wherein the total number of cells in each plurality is different between pluralities.

45. A fixed array of semi-solid microwells with pluralities of microwells physically partitioned from each other, wherein the microwells within a plurality comprise a non-uniform number of cells, and wherein one or more cells are overlaid with an extracellular matrix and a semi-solid matrix, optionally wherein total cells between pluralities is approximately uniform.

46. The fixed array of claim 45, wherein one or more cells are fixed in microwells by an overlay of a semi-solid matrix.

47. The fixed array of claim 45, wherein the overlay of a semi-solid matrix is an overlay of low melting point agarose.

48. The fixed array of any one of claims 45-47, wherein each plurality comprises about 50, about 100, about 200 or about 500 microwells.

49. The fixed array of any one of claims 45-48, wherein the cells are adherent cells.

50. The fixed array of any one of claims 45-49, wherein the cells are non-adherent cells.

51. The fixed array of any one of claims 45-50, wherein the semi-solid microwells comprise a semi-solid matrix and culture medium.

52. The fixed array of claim 51, wherein the semi-solid matrix is normal melting point agarose.

53. The fixed array of any one of claims 45-52, wherein the fixed array is immersed in culture medium.

54. The fixed array of any one of claims 45-53, wherein microwells within a plurality comprise 0-7 cells per microwell.

55. The fixed array of any one of claims 45-54, further comprising a cell membrane permeable DNA-specific fluorescent dye.

56. The fixed array of any one of claims 45-55, wherein the cells have not been lysed.

57. A fixed array of semi-solid microwells with pluralities of microwells physically partitioned from each other, and a cell membrane-permeable DNA-specific fluorescent dye, wherein the microwells within a plurality comprise a non-uniform number of non-lysed cells, wherein total cells between pluralities is approximately uniform.
